(19) United States
(12) Patent Application Publication
(10) Pub. No.: US 2012/0029412 A1
(43) Pub. Date: Feb. 2, 2012

(54) INTERNAL AND EXTERNAL DISC SHUNT ALLEVIATE BACK PAIN

(76) Inventors: Jeffrey E. Yeung, San Jose, CA (US); Teresa T. Yeung, San Jose, CA (US)

(21) Appl. No.: 13/138,071

(22) PCT Filed: Jan. 3, 2011
(86) PCT No.: PCT/US11/00007

§ 371 Date: Jun. 30, 2011

Related U.S. Application Data
(60) Provisional application No. 61/335,140, filed on Jan. 2, 2010.
(60) Provisional application No. 61/399,088, filed on Jul. 6, 2010.

(51) Int. Cl. A61M 5/00

(52) U.S. Cl. 604/8

(57) ABSTRACT

The intervertebral disc is avascular. Nutrients and waste are diffused through adjacent vertebral bodies into the disc. As we age, calcified layers form between the disc and vertebral bodies, blocking diffusion of nutrients, oxygen and pH buffer in blood. Under anaerobic conditions, lactic acid is produced, irritating nerve endings and causing nonspecific pain. In addition, the disc begins to starve and flatten. The weight shifts abnormally from disc to the facet joints causing strain and back pain. Shunt coils are formed and spiraled over the distal shaft of a twistable needle, then deployed into the nucleus of a degenerated disc by a sliding sleeve. The coils serve as an internal shunt, drawing nutrients, oxygen and buffering solute from the superior and inferior diffusion zones to neutralize lactic acid in the mid layer of the degenerated disc. The coils also serve as a bulking agent within the repaired disc to sustain compression and reduce facet loading and segmental instability. The end strands of the shunt coils can also extend from the disc to draw blood plasma from muscle or bodily circulation to expedite neutralization of lactic acid and rebuild disc matrix for pain relief and disc regeneration.

### FIELD OF INVENTION

Diffusion of nutrients, oxygen and pH buffer into avascular intervertebral disc is limited to the depths of diffusion zones near superior and inferior endplates. Lactic acid produced anaerobically in the mid layers of the nucleus can leak out of the disc and cause persistent back pain. This invention relates to devices drawing nutrients, oxygen and pH buffer from diffusion zones supplied by capillaries in the endplates to neutralize the lactic acid to relieve back pain. The device also serves as a bulking agent within the degenerated disc to reduce strain and pain of the facet joints. Furthermore, strands of the device can extend from the disc into muscle to draw additional nutrients, oxygen and pH buffer to neutralize the acid and regenerate the disc.

### BACKGROUND

Chronic back pain is an epidemic. Nerve impingement is not seen by CT or MRI in about 85% of back pain patients [Deyo R A, Weinstein J N: Low back pain, N Eng J Med, 344(5) February, 363-370, 2001. Boswell M V, et. al.: Interventional Techniques: Evidence-based practice guidelines in the management of chronic spinal pain, Pain Physician, 10:7-111, ISSN 1533-3159, 2007]. In fact, lumbar disc prolapse, protrusion, or extrusion account for less than 5% of all low back problems, but are the most common causes of nerve root pain and surgical interventions (Manchikanti L, Derby R, Benyamin R M, Helm S, Hirsch J A: A systematic review of mechanical lumbar disc decompression with nucleoplasty, Pain Physician; 12:561-572 ISSN 1533-3159, 2009). The cause of chronic back pain in most patients has been puzzling to both physicians and patients.

Studies indicate back pain is correlated with high lactic acid in the disc. Leakage of the acid causes acid burn and persistent back pain. In addition, as the disc degenerates and flattens, the compressive load is shifted from the flattened disc to facet joints, causing strain and pain. Both lactic acid burn and strain of the facet joints are not visible under CT or MRI.

### SUMMARY OF INVENTION

A disc shunt delivery device contains a needle, a sleeve with a snagging point and a shunt strand extending from a lumen of the needle and draping outside the sleeve and needle with a beveled tip. As the needle is twisted or rotated, the beveled tip catches and winds the outside shunt strand to spiral around the needle. The sleeve slides over the needle, using the snagging point to snag and dislodge the spiraled shunt strand from the needle into the disc. Spiraling and dislodgement of coiled shunt strands can be repeated to build an internal disc shunt near one or both endplates to draw nutrients, oxygen and buffering solute supplied through the endplates to neutralize lactic acid and relieve back pain. The internal disc shunt also serves as a cushion or bulking agent within the disc to reduce load, strain and pain in facet joints.

One or more strands of the internal disc shunt can be extended outside the disc into muscle or bodily circulation to draw additional nutrients, oxygen and/or pH buffer solute into the disc, forming an internal and external disc shunt.

### REFERENCE NUMBERS

100 Intervertebral disc
100A L5-S1 disc
100B L4-5 disc
100C L3-4 disc
101 Needle
102 Dull external edge of the distal end of the needle
103 Guide wire or tube
104 Filament of disc shunt
105 Endplate
106A Superior diffusion zone
106B Inferior diffusion zone
107 Capillaries
108 Calcified layers
109 Dip stick
110 Beveled or indented distal end of the sleeve
111 Lumen of the cannula needle
114 Annular delamination
115 Epiphysis
116 Lumen for guide wire
118 Nerve
119 Epidural space
121 Fissure
122 Gel or foam internal disc shunt
123 Spinal cord
124 Pores of sponge shunt
126 Main shunt
126A U-section, bent section, distal portion, or distal section of the main shunt
126B Second end-strand or portion of the main shunt
126C First end-strand or portion of the main shunt
127 Shunt sheath, wrapper or cover layer
128 Nucleus pulposus
129 Facet joint
130 Handle of needle
131 Nutrients, oxygen and pH buffering solute
132 Handle of sleeve
133 Transverse process
134 Spinous process
135 Lamina
140 Ilium
142 Superior articular process
143 Inferior articular process
152 Puncture site
153A Marker showing orientation of the sharp needle Quincke tip
153B Marker showing orientation of the snagging point of sleeve
153C Marker showing orientation of cannula Quincke tip
159 Vertebral body
160 Biosynthetic product or molecule
161 Fluid flow
162 Lactic acid
163 Contrast agent
184 Nucleus hole
193 Muscle
194 Spinal nerve root
195 Posterior longitudinal ligament
220 Sleeve
221 Snagging point, tip or edge of the sleeve
230 Cannula needle
231 Quincke tip of the cannula needle
232 Dull external edge of the cannula needle
233 Dull or rounded inner wall of the cannula needle
268 Lumen of the sleeve
269 Lumen of the needle
270 Handle of the cannula needle
271 Proximal protrusion of cannula handle
272 Distal protrusion of cannula handle
276A Syringe
276B Contrast injecting needle
277 Cell
278 Pedicle
279 Sleeve pusher
310 Quincke sharp tip of the needle
360 Sleeve pushing slot or opening
362 Sleeve pushing stop
363 Sleeve pushing hinge
368 Blade-like inner wall of the needle
369 Damaged portion of the shunt
370 Dull or rounded inner wall of the needle
373 Linked or attached shunt
373A Linked U-section, linked bent section or linked distal section of the linked shunt
373B First linked end strand or portion
373C Second linked end strand or portion
378 Annulus or annular layer
460 Pull line
461 Retainer or holder of the shunt stands
462 Fold or crease on the pull line
463 Knot on the pull line
492 Proximal opening of bi-handle holder
493 Bi-handle holder
494 Cavity of bi-handle holder
495 Distal wall of bi-handle holder
496 Distal opening of bi-handle holder
497 Proximal wall of bi-handle holder
498 Distal protrusion of sleeve handle
499 Proximal protrusion of sleeve handle
500 Distal protrusion of needle handle
501 Proximal protrusion of needle handle
502 Gripping or friction ridges of needle handle
503 Needle-sleeve spacer
504 Kambin's triangle
505 Skin
506 Sleeve-cannula spacer
507A Distal wall of sleeve-cannula spacer
507B Distal opening of sleeve-cannula spacer
508A Proximal wall of sleeve-cannula spacer
508B Proximal opening of sleeve-cannula spacer
509 Cavity of sleeve-cannula spacer
510 Tri-handle holder
511A Distal wall of tri-handle holder
511B Distal opening of tri-handle holder
512A Proximal wall of tri-handle holder
512B Proximal opening of tri-handle holder
513 Cavity of tri-handle holder
514 Esophagus
515 Larynx or trachea

### DESCRIPTION OF THE DRAWINGS

FIG. 1 shows a longitudinal view of a healthy spinal segment with nutrients 131 supplied by capillaries 107 at the endplates 105 to feed the cells within the disc 100.

FIG. 2 shows a graph of distance from endplate of a disc versus oxygen concentration.

FIG. 3 shows calcified layers 108 accumulated at the endplates 105, blocking diffusion of nutrient/oxygen 131 from capillaries 107, forming and leaking lactic acid 162 to nerve 118.

FIG. 4 shows leakage of lactic acid 162, burning or irritating the spinal nerve 194.

FIG. 5 depicts diagnostic discography by flushing lactic acid from disc 100 with contrast agent 163 to sensory nerve 118 to confirm pain.

FIG. 6 shows a hole or vacuole 184 in the disc 100.

FIG. 7 shows load transfer from the flattened and degenerated disc 100 to facet joint 129.

FIG. 8 depicts swaying of a vertebral body 159 above a disc 100 with low-swelling pressure.

FIG. 9 depicts spinal instability from the low-pressure disc 100, straining and wearing the facet joints 129.

FIG. 10 shows portions of main shunt 126, linked shunt 373, needle 101 and sleeve 220 for treating discogenic and facet pain.

FIG. 11 shows a fluoroscopic anterior-posterior view of the needle 101, about half way past pedicles 278, entering into the disc 100 space.

FIG. 12 shows a fluoroscopic lateral view of the needle 101 entering into the disc 100 space, but not into the epidural space 119.

FIG. 13 shows entry of the needle 101 and shunt strands 126, 373 through skin 505, muscle 193 and Kambin's triangle 504 of the degenerated disc 100.

FIG. 14 shows a needle handle 130, sleeve handle 132 and a bi-handle holder 493 to facilitate disc 100 puncturing.

FIG. 15 shows twisting or rotation of the beveled needle 101 to wind or spiral the shunt strands 126B, 373B, 373C on the distal shaft of the needle 101.

FIG. 16 shows a sleeve pusher 279 for inserting between the sleeve handle 132 and needle handle 130 to advance the sleeve 220.

FIG. 17 shows a snagging point 221 on the distal end of the advancing sleeve 220 to snag, catch, hook, connect, push or engage the spiraled shunt strands 126B, 373B, 373C.

FIG. 18 shows progressive advancement of the sleeve 220 to dislodge, push or strip the spiraled shunt strands 126B, 373B, 373C off the needle 101.

FIG. 19 shows that the snagging point 221 slides parallel to the needle 101 to deploy or dislodge the spiraled shunt strands 126B, 373B, 373C within the disc.

FIG. 20 shows slight withdrawal of the needle 101 to expose a new strand 126C from the lumen of the needle 101. The needle 101 will then advance, so distal tips of the needle 101 and sleeve 220 are generally aligned as shown in FIG. 19.

FIG. 21 shows withdrawal of the sleeve 220 and coiling of shunt strands 126B, 373B, 373C over strand 126C extending from the lumen 269 of the needle 101.

FIG. 22 shows subsequent twisting of the needle 101 to spiral another length of shunt strands 126B, 373B, 373C on the distal shaft of the needle 101.

FIG. 23 shows substantial repetitive spiraling of disc shunts 126, 373 within the degenerated disc 100, before cutting shunt strands 126B, 126C, 373B and 373C.

FIG. 24 shows the shunt strands 126B, 373B, 373C being reeled under the skin 505 by adding more spiraled shunt strands 126, 373 into the disc 100. A dip stick 109 is used to check the depth of the shunt strand 126C within the needle 101.

FIG. 25 shows the internal shunts 126, 373 within the disc 100, and external shunt strands 126B, 126C, 373B, 373C drawing plasma from the muscle 193 into the disc 100.

FIG. 26 shows the internal shunt 126, 373 drawing nutrients/oxygen/buffer 131 from superior 106A and inferior 106B diffusion zones, and the external shunt 126, 373 drawing nutrients/oxygen/buffer 131 from muscle 193 into the disc 100.

FIG. 27 shows thickening of the repaired disc 100 by the spiraled internal disc shunts 126, 373 to reduce load, strain and pain of the facet joints 129.

FIG. 28 shows an internal disc shunts 126, 373 entirely spiraled, coiled, knotted or deployed within the disc 100, reaching one or more diffusion zones 106A, 106B.

FIG. 29 shows that the internal shunts 126, 373 reach, absorb and/or draw nutrients 131 from the superior 106A and/or inferior 106B diffusion zones into the mid layers of the disc 100.

FIG. 30 depicts compression on the internal shunts 126, 373, squeezing nutrients 131 absorbed in the shunts 126, 373 to mid layers and other portion of the disc 100.

FIG. 31 depicts relaxation or expansion of the internal shunt 126, 373, drawing or absorbing nutrients 131 from the superior 106A and inferior 106B diffusion zones.

FIG. 32 shows injection of a gel or foam shunt 122, capable of drawing nutrients 131 from the superior 106A and/or inferior 106B diffusion zones into the mid layers of the disc 100.

FIG. 33 shows shielding of L5-S1 disc 100A, L4-5 disc 100B by the ilium 140, blocking entry of the straight needle 101.

FIG. 34 shows ilium shielding of the lower lumbar disc 100, preventing needle 101 entry into the nucleus of the disc 100.

FIG. 35 shows curvatures of the needle 101 and sleeve 220 deployed from a straight and rigid cannula needle 230 into the nucleus 128 of the intervertebral disc 100.

FIG. 36 shows the curved needle 101 and sleeve 220 with shunt strands 126B, 373B and 373C draped outside the needle 101, sleeve 220 and cannula needle 230.

FIG. 37 shows the handle of the needle 130, handle of the sleeve 132, handle of the cannula needle 270, sleeve-cannula spacer 506 and a tri-handle holder 510.

FIG. 38 shows the resiliently straightened curved needle 101 and sleeve 220 within the cannula needle 230 with a guide wire 103 leading into the disc 100.

FIG. 39 shows a mid-longitudinal view of a naturally occurring blade-like inner wall 368 of the needle 101, cutting the U-section 126A of the main shunt 126 during tissue puncturing.

FIG. 40 shows a rounded, blunt or dull inner wall 370 of the needle 101, supporting without cutting the U-section 126A of the main shunt 126.

FIG. 41 shows a rounded, blunt or dull inner wall 233 of the cannula needle 230 to prevent cutting the U-section 126A of the main shunt 126.

FIG. 42 shows two snagging points or tips 221 of the sleeve 220 for engaging and dislodging the spiraled strands 126B, 373B, 373C from the distal shaft of the needle 101.

FIG. 43 shows multiple snagging points or tips 221 of the sleeve 220.

FIG. 44 shows a single snagging point or tip 221 of the sleeve 220.

FIG. 45 shows a longitudinal view of the spiraled strands 126B, 373B, 373C, the needle 101 and the sleeve 220 with snagging points 221 made by beveling the inner wall of the sleeve 220.

FIG. 46 shows braided filaments 104 to form the disc shunt strands 126, 373.

FIG. 47 shows woven filaments 104 to form the disc shunt strands 126, 373.

FIG. 48 shows knitted filaments 104 to form the disc shunt strands 126, 373.

FIG. 49 depicts a slanted cut of the disc shunt strands 126, 373, showing the slanted orientations of filaments 104 relative to the length-wise shunt strands 126, 373.

FIG. 50 shows cross-sections of filaments 104 oriented parallel to shunt strands 126, 373, wrapped, encircled or enveloped by a sheath or cover 127.

FIG. 51 shows cross-sections of tubular filaments 104 oriented parallel to the shunt strands 126, 373, wrapped, encircled or enveloped by a sheath or cover 127.

FIG. 52 shows a disc shunt strand 126 or 373 made with sponge or foam with pores 124.

FIG. 53 shows a section of the disc shunt strand 126, 373 transporting and supplying nutrients 131 to cells 277 to produce biosynthetic products 160.

FIG. 54 shows fluid flowing 161 into the disc 100 due to increased osmolarity from newly made biosynthetic products 160 using the continual supply of nutrients 131.

FIG. 55 shows injection of nutrients 131 and/or cells 277 into the internal and external shunted disc 100 to expedite production of biosynthetic products 160.

FIG. 56 shows a misguided needle 101 and sleeve 220 delivering shunt strands 126B, 373B, 373C under the skin 505 of a neck.

FIG. 57 shows needle 101 withdrawal for redirecting the needle 100, but pre-maturely deploying the shunt strands 126B, 126C, 373B, 373C under skin 505.

FIG. 58 shows pull lines 460 threaded through the proximal ends of the shunt strands 126B, 373B, 373C, and the shunt strand 126C within the needle 101.

FIG. 59 shows a retainer 461 holding the shunt strands 126B, 373B, 373C for attachment to the pull line 460.

FIG. 60 depicts a crease 462 formed on the pull line 460 during tension pulling on the shunt strands.

FIG. 61 depicts release of tension from the crease-resistant pull line 460 to facilitate pull line 460 withdrawal from the shunt strands.

FIG. 62 shows the pull line 460 attached to the shunt strands 126B, 373B, 373C and extending above the skin 505 to assist needle 101 withdrawal and redirecting.

### DETAILED DESCRIPTION OF THE EMBODIMENTS

Intervertebral discs are avascular (no blood vessels). Nutrients, oxygen and pH buffer 131 essential for disc cells are supplied by the capillaries 107 in the vertebral bodies 159 and diffused from the superior and inferior endplates 105 into the disc 100, as shown in FIG. 1. Normal blood pH is tightly regulated between 7.35 and 7.45, mainly by the pH buffering bicarbonate dissolved in blood plasma diffused through the superior and inferior endplates 105 into the disc 100.

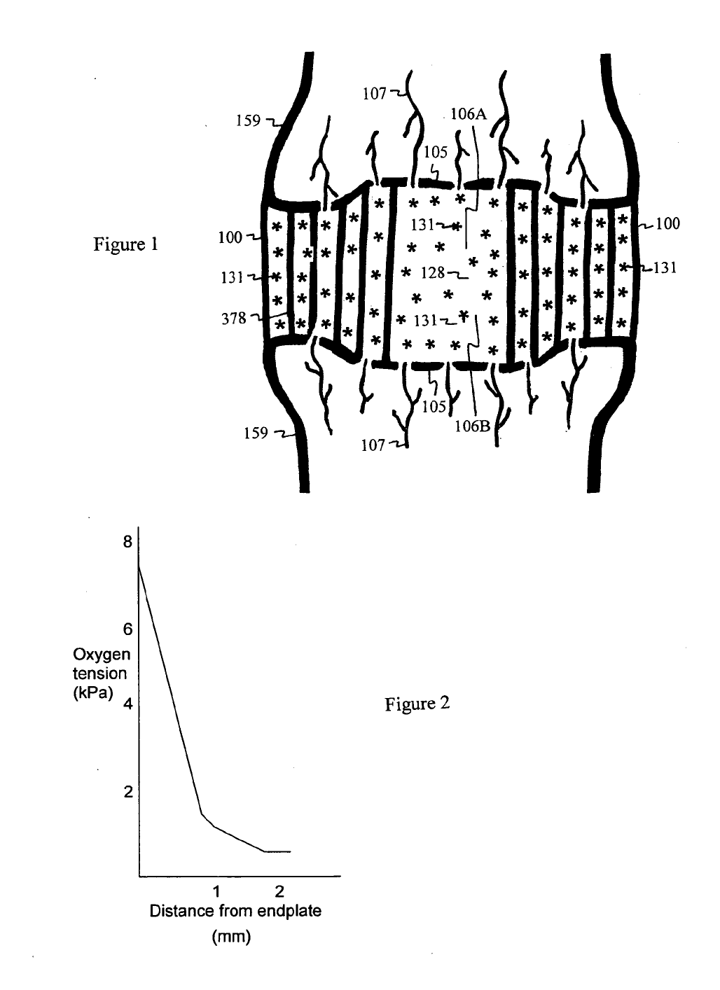

However, depth of diffusion is shallow into thick human discs 100. The calculated depth of oxygen diffusion from the endplates 105 is summarized in FIG. 2 (Stairmand J W, Holm S, Urban J P G: Factor influencing oxygen concentration gradients in disc, Spine, Vol. 16, 4, 444-449, 1991).

Similarly, calculated depths of glucose diffusion are less than 3 mm from superior and inferior endplates (Maroudas A, Stockwell R A, Nachemson A, Urban J: Factors involved in the nutrition of the human lumbar intervertebral disc: Cellularity and diffusion of glucose in vitro, J. Anat., 120, 113-130, 1975). Nearly all animals have thin discs; depths of diffusion of nutrients and oxygen seem to be sufficient. Lumbar discs of a large sheep weighing 91 kg (200 pounds) are less than 4 mm thick. However, human lumbar discs are about 7-12 mm thick. Mid layers of our thick discs are highly vulnerable to severe nutritional and oxygen deficiency.

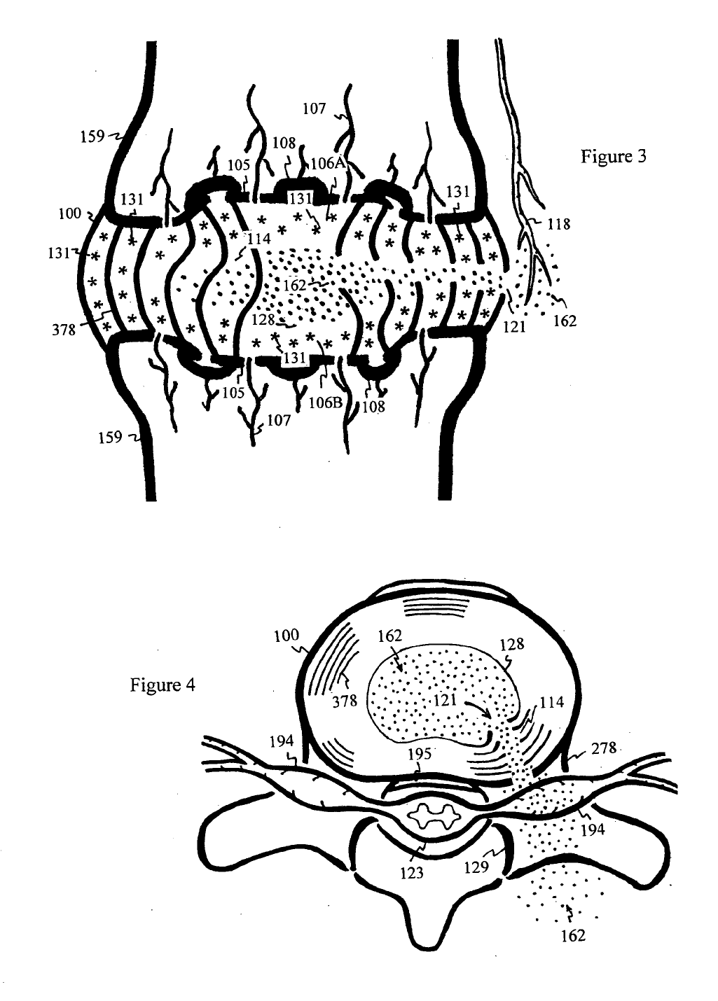

As we age, calcified layers 108 form and accumulate at the endplates 105, blocking capillaries 107 and further limiting the depth of diffusion of nutrients/oxygen/pH buffer 131 into the disc 100, as shown in FIG. 3. Cell death, matrix degradation and lactic acid 162 accumulation due to starvation and anaerobic conditions are common in the mid layer of the avascular discs 100. Degradation of glycosaminoglycans may provide sugars to fuel the production of lactic acid 162. [Urban J P, Smith S, Fairbank J C T: Nutrition of the Intervertebral Disc, Spine, 29 (23), 2700-2709, 2004. Benneker L M, Heini P F, Alini M, Anderson S E, Ito K: Vertebral endplate marrow contact channel occlusions & intervertebral disc degeneration, Spine V30, 167-173, 2005. Holm S, Maroudas A, Urban J P, Selstam G, Nachemson A: Nutrition of the intervertebral disc: solute transport and metabolism, Connect Tissue Res., 8(2): 101-119, 1981].

When glycosaminoglycans diminish, water content and swelling pressure of the nucleus pulposus 128 decrease. The nucleus 128 with reduced swelling pressure can no longer distribute forces evenly against the circumference of the inner annulus 378 to keep the annulus bulging outward. As a result, the inner annulus 378 sags inward while the outer annulus 378 bulges outward, creating annular delamination 114 and weakened annular layers 378, possibly initiating fissure 121 formation depicted in FIGS. 3 and 4.

High lactic acid content in discs correlates with back pain. In fact, dense fibrous scars and adhesions, presumably from lactic acid 162 burn, can be found around nerve roots 194 during spinal surgery [Diamant B, Karlsson J, Nachemson A: Correlation between lactate levels and pH of patients with lumbar rizopathies, Experientia, 24, 1195-6, 1968. Nachemson A: Intradiscal measurements of pH in patients with lumbar rhizopathies. Acta Orthop Scand, 40, 23-43, 1969. Keshari K R, Lotz J C, Link T M, Hu S, Majumdar S, Kurhanewicz J: Lactic acid and proteoglycans as metabolic markers for discogenic back pain, Spine, Vol. 33(3):312-317, 2008].

Under anaerobic condition within the mid layer, lactic acid 162 is produced and leaked from the nucleus 128 through fissure 121 to burn surrounding nerves 118 causing persistent back pain, as depicted in FIG. 3. Colored drawings in the U.S. Provisional Application 61/399,088, Alleviate back pain by expanding the diffusion zones, filed on Jul. 6, 2010 by Jeffrey Yeung and Teresa Yeung show superior and inferior diffusion zones near the calcified endplates and lactic acid zone in the mid layer of the degenerated disc. Similar black and white drawing is depicted in FIG. 3.

Some patients experience leg pain without visible spinal nerve impingement under MRI or CT. Lactic acid 162 can leak from the nucleus 128 through fissures 121 to spinal nerves 194, causing leg pain as depicted in FIG. 4. Leg pain without visible impingement is commonly called chemical radiculitis.

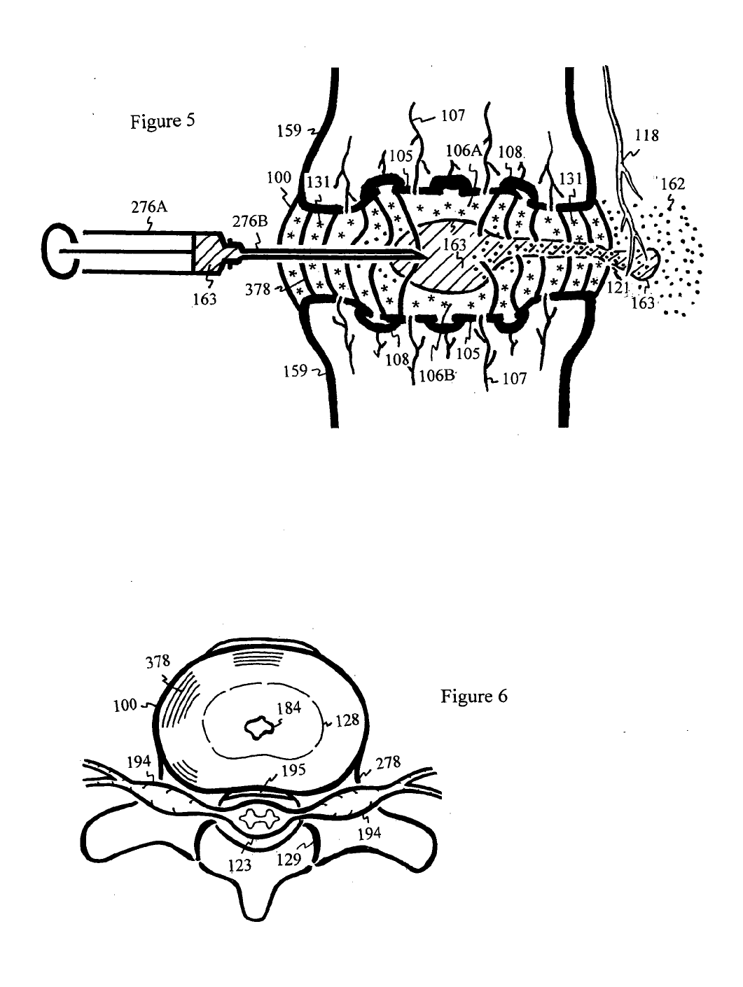

Discography is a common diagnostic technique for identifying or confirming a painful disc 100 before surgical intervention. Intradiscal injection of an X-ray contrast 163 flushes the lactic acid 162 from the nucleus 128 through fissure 121 to adjacent nerve 118, causing instant and excruciating pain, as shown in FIG. 5. For normal or non-painful discs, discography with mild injection pressure is nearly painless.

Composition Change of the Intervertebral Discs (Approximation)

| | | | % Change from |
|---|---|---|---|
| | Normal Discs | Painful Discs | Normal Discs |
| Glycosamino- | 27.4 ± 2.4% | 14.1 ± 1.1% | −48.5% |
| glycans | | | |
| Collagen | 22.6 ± 1.9% | 34.8 ± 1.4% | +54% |
| Water content | 81.1 ± 0.9% | 74.5 ± 1% | −8.1% |
| Acidity | pH 7.14 | pH 6.65-5.70 | [H⁺]: +208% |
| | [H⁺]: 7.20 × 10⁻⁸ | [H⁺]: 2.23 × 10⁻⁷ | to +2,661% |
| | | to 2.00 × 10⁻⁶ | |

(Reference: Kitano T, Zerwekh J, Usui Y, Edwards M, Flicker P, Mooney V: Biochemical changes associated with the symptomatic human intervertebral disk, Clinical Orthopaedics and Related Research, 293, 372-377, 1993. Scott J E, Bosworth T R, Cribb A M, Taylor J R: The chemical morphology of age-related changes in human intervertebral disc glycosaminoglycans from cervical, thoracic and lumbar nucleus pulposus and annulus fibrosus. J. Anat., 184, 73-82, 1994. Diamant B, Karlsson J, Nachemson A: Correlation between lactate levels and pH of patients with lumbar rizopathies, Experientia, 24, 1195-1196, 1968. Nachemson A: Intradiscal measurements of pH in patients with lumbar rhizopathies, Acta Orthop Scand, 40, 23-43, 1969.)

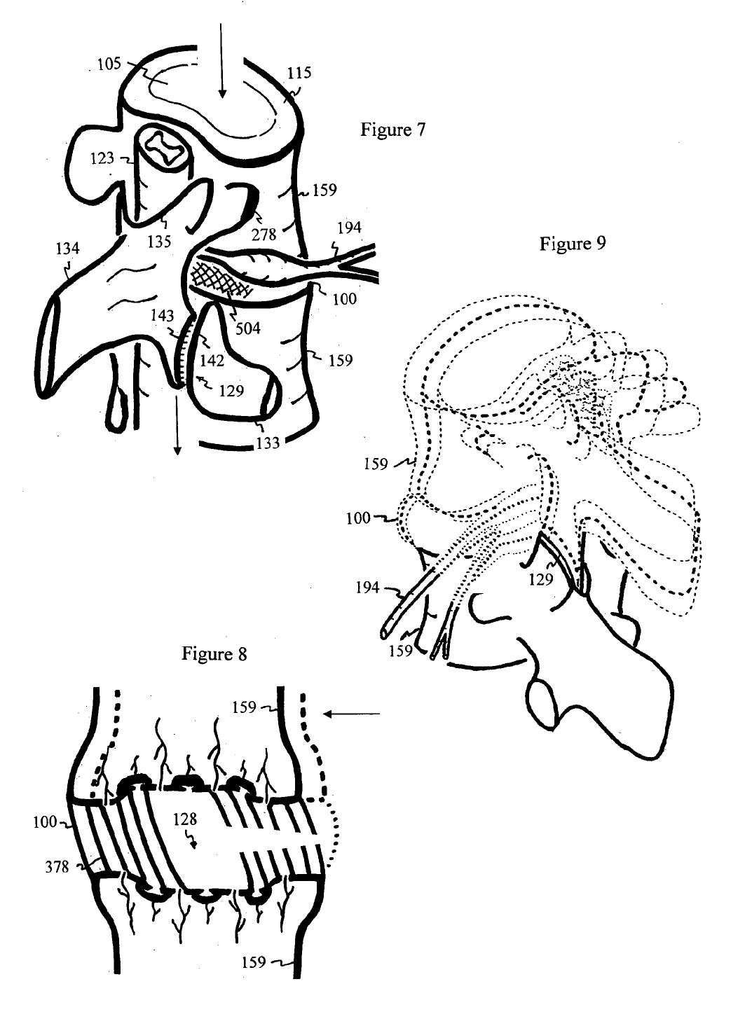

Disc cells can survive without oxygen, but will die without glucose. The central area in the mid layer of the disc 100 is most vulnerable to glucose deficiency and cell death. Holes or vacuoles 184 can be found during dissection of cadaveric discs 100, as shown in FIG. 6. Nuclei pulposi 128 of degenerated discs 100 are usually desiccated, with reduced swelling pressure and decreased capability to sustain compressive loads. The compressive load is thus transferred to the facet joints 129, pressing the inferior articular process 143 against the superior articular process 142 of the facet joint 129, causing strain, wear and/or pain as shown in FIG. 7 (Dunlop R B, Adams M A, Hutton W C: Disc space narrowing and the lumbar facet joints, Journal of Bone and Joint Surgery—British Volume, Vol 66-B, Issue 5, 706-710, 1984).

A disc 100 with reduced swelling pressure is similar to a flat tire with flexible or flabby side walls. The vertebral body 159 above the soft or flabby disc 100 easily shifts or sways, as shown in FIG. 8. This is commonly called segmental or spinal instability. As shown in FIG. 9, the frequent or excessive movement of the vertebral body 159 strains the facet joints 129, which are responsible for limiting the range of segmental mobility. Patients with spinal instability often use their muscles to guard or support their spines to ease facet pain. As a result, muscle tension and aches arise, but are successfully treated with muscle relaxants. Spinal motions, including compression, torsion, extension, flexion and lateral bending, were measured before and after saline injection into cadaveric discs. Intradiscal saline injections reduced all spinal motions in the cadaveric study (Andersson G B J, Schultz A B: Effects of fluid on mechanical properties of intervertebral discs, J. Biomechanics, Vol. 12, 453-458, 1979).

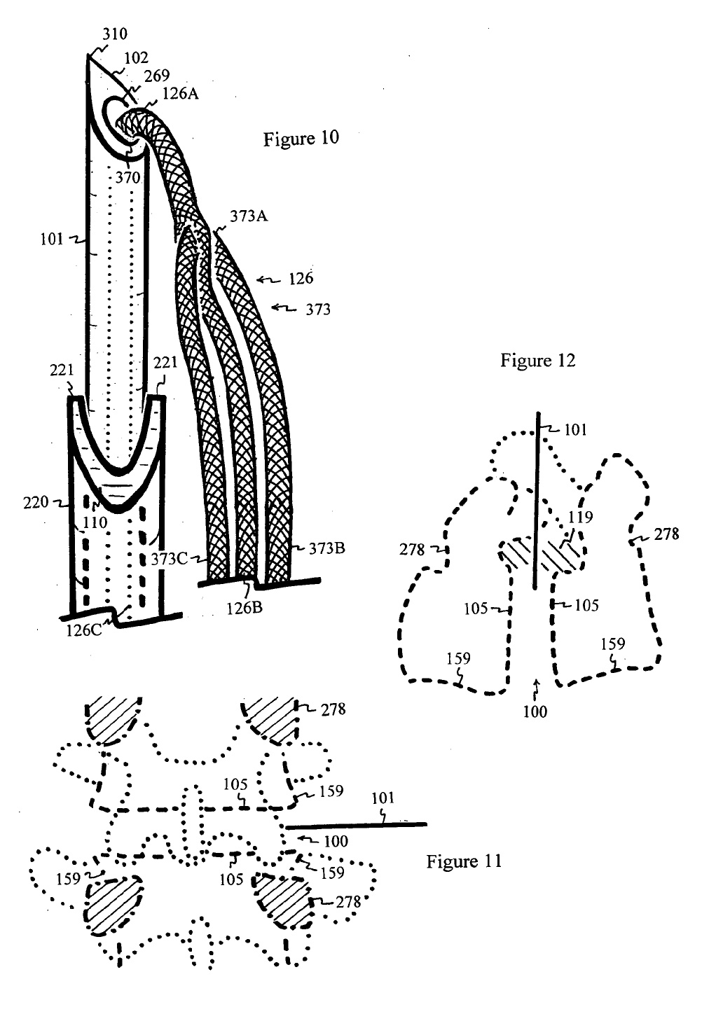

The shunt 126, 373 delivery needle 101 in FIG. 10 is made for tissue puncturing, not tissue cutting to prevent nerve injury. Unlike common needles with blade-like distal cutting edges, the shunt 126, 373 delivery needle 101 has a Quincke sharp tip 310 and dull external beveled edges 102. Similar to an awl, the shunt 126, 373 delivery needle 101 penetrates skin 505, muscle 193 and disc 100, gently pushing or deflecting the embedded blood vessels or spinal nerves 194 aside during penetration. The Quincke tip 310 can be called the beveled tip of the needle 101.

A main shunt strand 126 in FIG. 10 has two end-strands or portions 126C, 126B, and a main U-section, U-strand, bent section or distal section 126A. The first end-strand 126C is inserted into or through a lumen 269 of the needle 101. The U-section 126A extends from the lumen 269, draping the second end-strand 126B over the outer wall of the needle 101. A linked shunt strand 373 also has two linked end-strands or portions 373B, 373C, and a linked U-section, linked U-strand, or linked distal section 373A. The linked shunt 373 is attached to or threaded through the second end-strand 126B to form the linked U-strand 373A, the first linked end-strand 373B and second linked end-strand 373C. The main shunt strand 126 can be called the first shunt strand 126. The linked shunt strand 373 can be called the second shunt strand 373. The end-strand can be called shunt strand, end portion, 126C, 126B, 373B or 373C. The main U-section 126A can be called the U-shaped distal portion. The linked U-strand 373A can be called the linked U-shaped distal portion.

The delivery device of the shunt strands 126, 373 contains a sleeve 220, sized and configured to retain or house the needle 101. The length of the sleeve 220 is shorter than the length of the needle 101. The shunt strands 126B, 373B, 373C drape outside the sleeve 220 and needle 101. The sleeve 220 has two snagging points 221 at the distal end and a solid side-wall, capable of sliding length-wise over the needle 101 shaft. The snagging points 221 maintain a fixed distance from the outer wall of the needle 101. The fixed distance is less than the outer-diameter or thickness of the shunt strands 126A, 126B, 126C, 373A, 373B or 373C. In addition, the gap between the needle 101 and sleeve 220 is less than the outer-diameter or thickness of the shunt strands 126A, 126B, 126C, 373A, 373B or 373C. The gap is an inner diameter of the sleeve 220 minus an outer diameter of the needle 101, which should be less than the thickness of the shunt strands 126A, 126B, 126C, 373A, 373B or 373C. Therefore, the shunt strands 126A, 126B, 126C, 373A, 373B or 373C cannot be trapped between the snagging point 221 and needle 101 shaft. Furthermore, the sleeve 220 wall thickness is preferred to be at least a seventh of the thickness of the shunt strands 126A, 126B, 126C, 373A, 373B or 373C. Thus, the height of the snagging points 221 is sufficient to catch and dislodge the spiraled shunts 126, 373 from the distal shaft of the needle 101.

Kambin's Triangle 504 shown in FIG. 7 is a posterior-lateral area through which a needle can access a lumbar disc 100 safely. Similar to needle entry for discography, the shunt 126, 373 delivery needle 101 is guided by a fluoroscope (X-ray), entering into a patient in prone position. FIG. 11 shows an anterior-posterior fluoroscopic view of the needle 101 entering into disc 100 space, between superior and inferior endplates 105. However, the anterior-posterior view does not show the ventral-dorsal position. Before passing the pedicle 278 midway, a lateral fluoroscopic view depicted in FIG. 12 must be taken to ensure the needle 101 is not too dorsal, entering into the epidural space 119. FIG. 12 depicts the lateral fluoroscopic view, showing the needle 101 tip is ventral to the epidural space 119, safely entering into the mid layer of the disc 100.

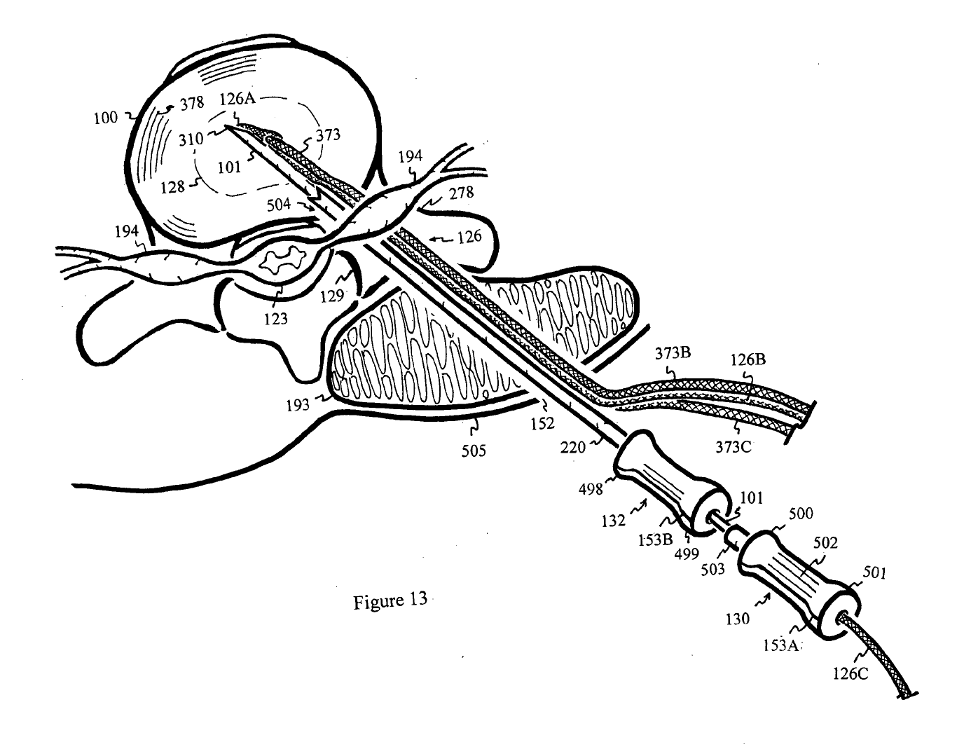

In literature, sizable disc puncturing or laceration causes disc degeneration. The shunts 126, 373 delivery device is self-sealing, as shown in FIG. 13. The shunt strands 126B, 373B, 373C outside the needle 101 and sleeve 220 are pressed against the wall of the needle 101 and sleeve 220, and squeezed into the annulus 378 through a very small punctured hole. After withdrawal of the needle 101 and sleeve 220, the shunt strands 126B, 126C 373B, 373C seal the needle tract within the annulus 378 to prevent or minimize the loss of hydrostatic pressure of the disc 100, as a press-fitted implant.

In sheep and human clinical study, the outer diameters of the needle 101 and sleeve 220 are only 1.00 and 1.27 mm respectively. The outer diameter of each shunt strand 126B, 126C, 373B or 373C is about 0.55 mm. Diameter of combined shunt strands 126B 126C, 373B, 373C is about 2.10 mm to seal the needle tract in the disc 100.

FIG. 13 shows initial entry of the needle 101 and shunt strands 126, 373 through skin 505, muscle 193 and Kambin's triangle 504 of the degenerated disc 100. Skin 505 of the puncture site 152 can be superficially cut with a scalpel to ease needle 101 puncture. During disc 100 puncturing, the Quincke sharp tip 310 of the needle 101 is preferred facing or near the mid line of the body to minimize the possibility of nicking the spinal nerve 194 or scraping the superior or inferior endplate 105. A marker 153A on a needle handle 130 indicates orientation of the Quincke tip 310, about 45 degrees from the endplates 105. The needle handle 130 also contains gripping or friction ridges 502 to facilitate twisting or rotating of the needle 101. The needle handle 130 is spool shaped with proximal protrusion 501 and distal protrusion 500 to facilitate needle 101 withdrawal and advancement.

To avoid scrapping the superior or inferior endplate 105, the snagging point 221 is preferred staying away or about 45 degrees from the superior and inferior endplates 105. A marker 153B on a sleeve handle 132 shows orientations of the snagging points 221, as shown in FIG. 13. The sleeve handle 132 also contains a proximal protrusion 499 to facilitate sleeve 220 withdrawal, and a distal protrusion 498 to facilitate sleeve 220 advancement, as shown in FIG. 13. The U- or distal sections 126A, 373A are in the disc 100. The shunt strands 126C, 126B, 373B, and 373C are usually extending from the disc 100 into the muscle 193. For lumbar disc 100 repair, the shunt strands 126C, 126B, 373B, and 373C are preferred to be long, extending outside the skin 505.

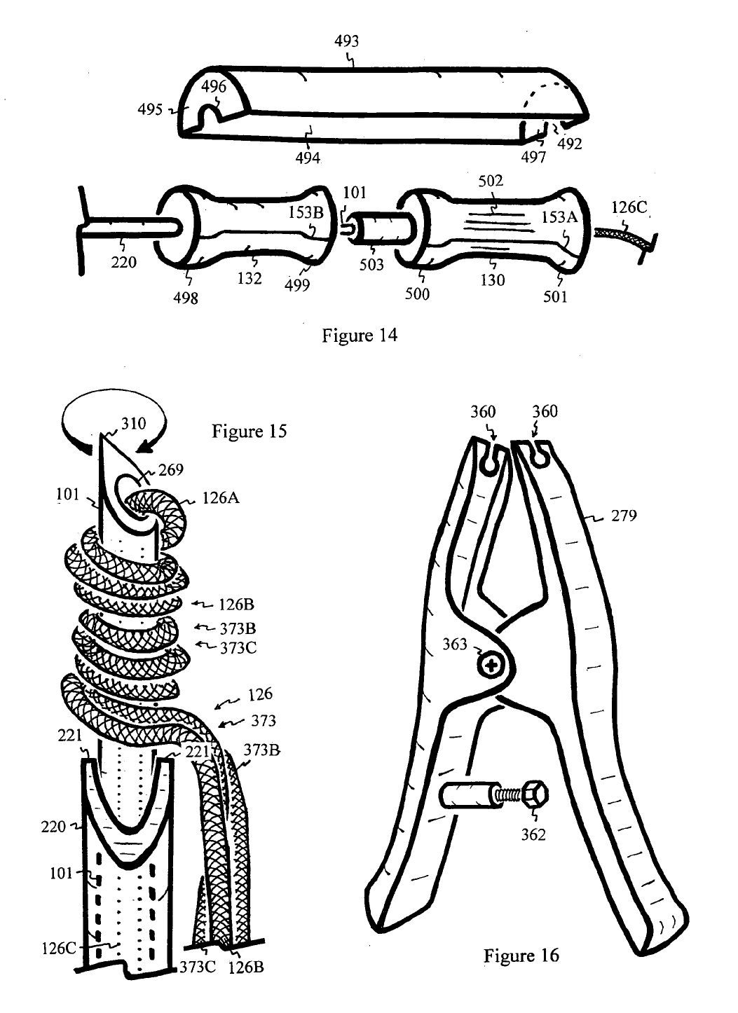

Both handles 130, 132 should be bound or linked together until the needle 101 is properly positioned within the degenerated disc 100. FIG. 14 shows a removable bi-handle holder 493 contains a bi-handle cavity 494 to house the needle handle 130 and the sleeve handle 132. The proximal wall 497 of the bi-handle holder 493 retains the needle handle 130; the proximal opening 492 of the proximal wall 497 arches over the shunt strand 126C. The distal wall 495 of the bi-handle holder 493 retains the sleeve handle 132; the distal opening 496 of the distal wall 495 arches over the sleeve 220. Binding the handles 130, 132 with the bi-handle holder 403 can further be fastened by a removable tie or band. The needle handle 130 and the sleeve handle 132 are separated by a needle-sleeve spacer 503 for insertion of a sleeve pusher 279.

After the needle 101 is positioned as shown in FIG. 13, the bi-handle holder 493 is removed. FIG. 15 shows twisting or rotation of the beveled needle 101 to wind, spiral, spool or coil the outside shunt strands 126B, 373B, 373C into a coiled or spiraled shunt strand, section or configuration on the distal shaft of the needle 101. Tension of the spiraled strands 126B, 373B, 373C can be felt on the needle handle 130 after about 3- to 7-needle 101 rotations. The U-section 126A contacting the inner wall at the lumen 269 of the needle 101 in FIG. 15 is vulnerable to damage or cutting. The inner wall of the needle 101 lumen 269 can be rounded or dulled by machining to prevent damage to the U-section 126A.

The main shunt 126 alone is sufficient to build the internal and/or external disc shunt 126. The linked shunt strand 373 adds bulk, size, cushion, filling or mass to the internal and/or external disc shunts 126, 373.

A sleeve pusher 279 contains a hinge 363, an adjustable stop 362, slots 360 and handles of the sleeve pusher 279, in FIG. 16. The adjustable stop 362 prevents excessive advancement of the sleeve 220 beyond the Quincke sharp tip 310 of the needle 101. For sleeve advancement, the needle handle 130 is held stationary. The slots 360 of the sleeve pusher 279 are inserted over the needle-sleeve spacer 503 between the sleeve handle 132 and needle handle 130. The needle handle 130 is held stationary, while using leverage of the sleeve pusher 279 to advance the sleeve 220 and dislodge the spiraled shunt strands 126B, 373B, 373C from the distal shaft of the needle 101 into the disc 100. During dislodgement, the strands 126B, 373B, 373C outside the skin 505 can be seen advancing into the body of the patient.

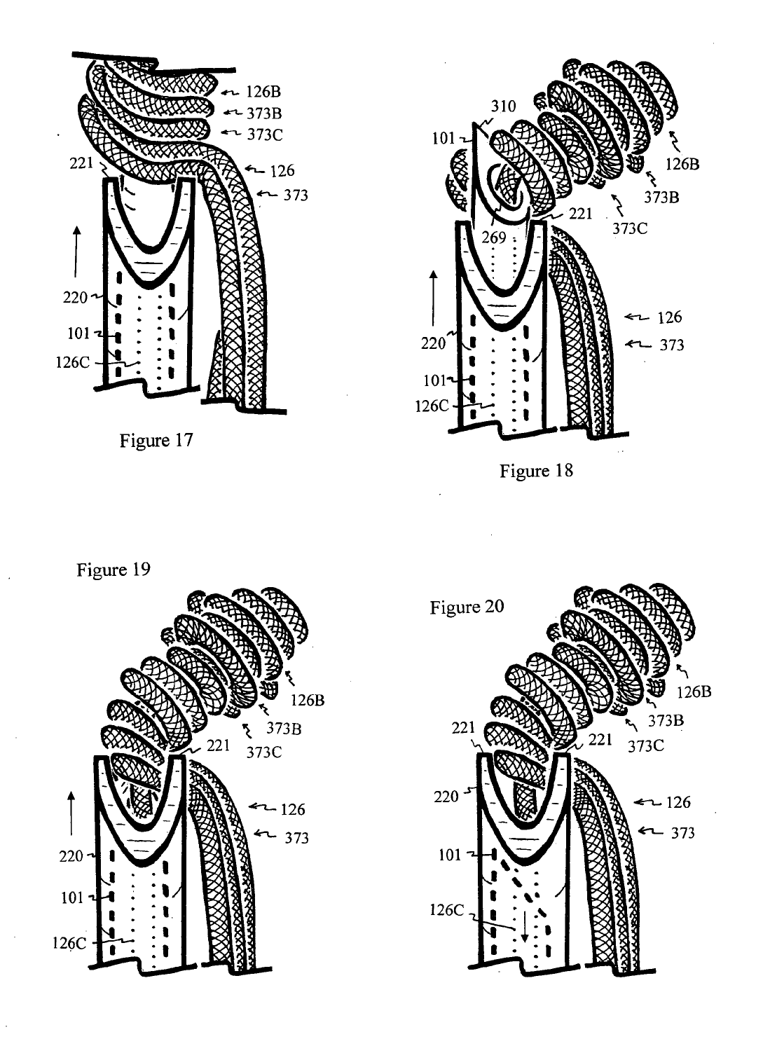

The snagging point 221 is preferred to be a sharp tip, edge or rim, protruding and maintaining a fixed distance, sliding parallel over the outer wall of the needle 101 shaft. The snagging point 221 on the distal portion of the advancing sleeve 220 snags, catches, hooks, pushes or engages the spiraled shunt strands 126B, 373B, 373C as shown in FIGS. 17-18.

Longitudinal advancement of the snagging points 221 of the sleeve 220 over the needle 101 creates minimal damage, disruption or opening to the annulus 378, for preserving hydrostatic pressure of the disc 100. The spiraled shunt strands 126B, 373B, 373C may have several layers coiled over the distal shaft of the needle 101. The sleeve 220 and the snagging points 221 slide over the needle 101 shaft to catch and push mainly the bottom layer of the shunt strands 126B, 373B, 373C. The needle 101 can be coated with a lubricant to ease dislodgement or deployment of shunt strands 126B, 373B, 373C. Furthermore, tension of the spiraled shunt strands 126B, 373B, 373C over the needle 101 shaft can be loosened by slightly counter turning the needle handle 130 before advancing the sleeve 220 to dislodge the spiraled shunt strands 126B, 373B, 373C. The sleeve 220 in FIGS. 17-20 has two snagging points 221, showing sequential dislodging, stripping or deploying of the spiraled shunt strands or section 126B, 373B, 373C from the distal shaft of the needle 101 into the degenerated disc 100.

During sleeve 220 advancement and strands 126B, 373B, 373C dislodging, the strand 126C is also pulled through the needle lumen 269 into the disc 100, as depicted in FIGS. 18-19. Furthermore, the spiraled strands 126B, 373B, 373C are wound, spiraled, coiled or spooled over the strand 126C. Therefore, the spiraled strands 126B, 126C, 373B, 373C are intertwined forming an inter-connected coil. Each shunt strand 126B, 126C, 373B or 373C is not easily expelled, extruded or migrated from the repaired disc 100. The coil or spiral of shunt strands 126B, 126C, 373B, 373C also serve as an anchor or large knot within the disc 100, too large to pass through the press-fitted needle tract.

For lumbar discs 100, initial spiraling of shunt strands or section 126B, 373B, 373C in FIG. 19 may not be sufficient to reach one or both superior 106A and inferior 106B diffusion zones. Additional spiraling and deployment of shunt strands are required to build the internal disc shunt 126, 373 and a bulking mass within the nucleus 128 to relieve pain from lactic burn and facet joint 129 loading. It is prudent to check positions of the needle 101 and sleeve 220 through fluoroscopic views after each deployment of spiraled strands 126B, 373B, 373C.

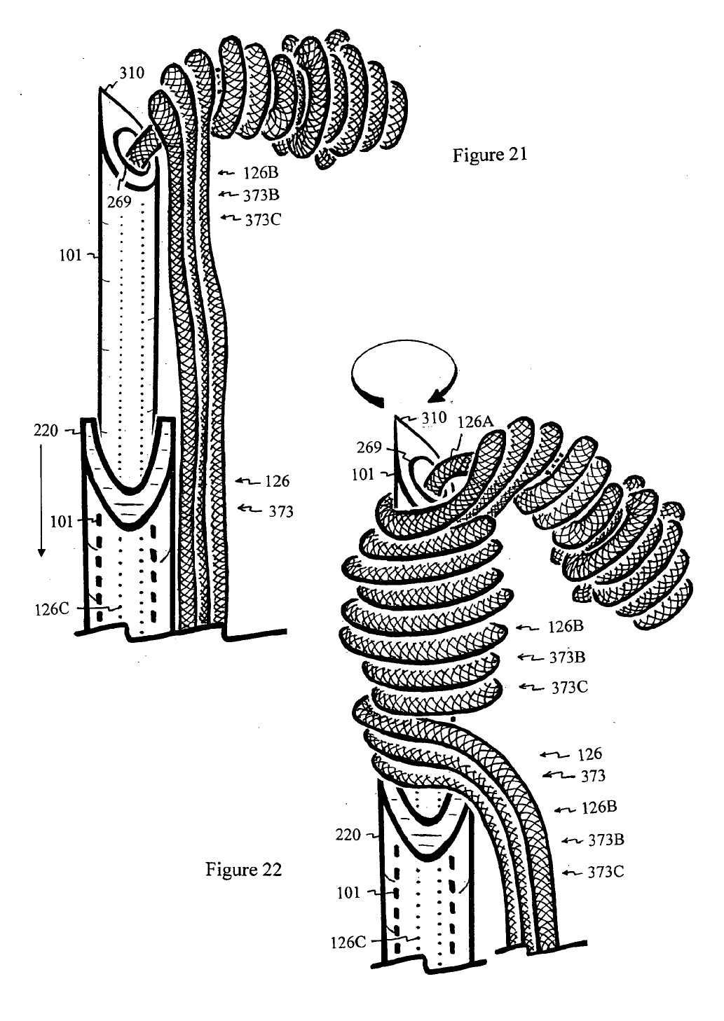

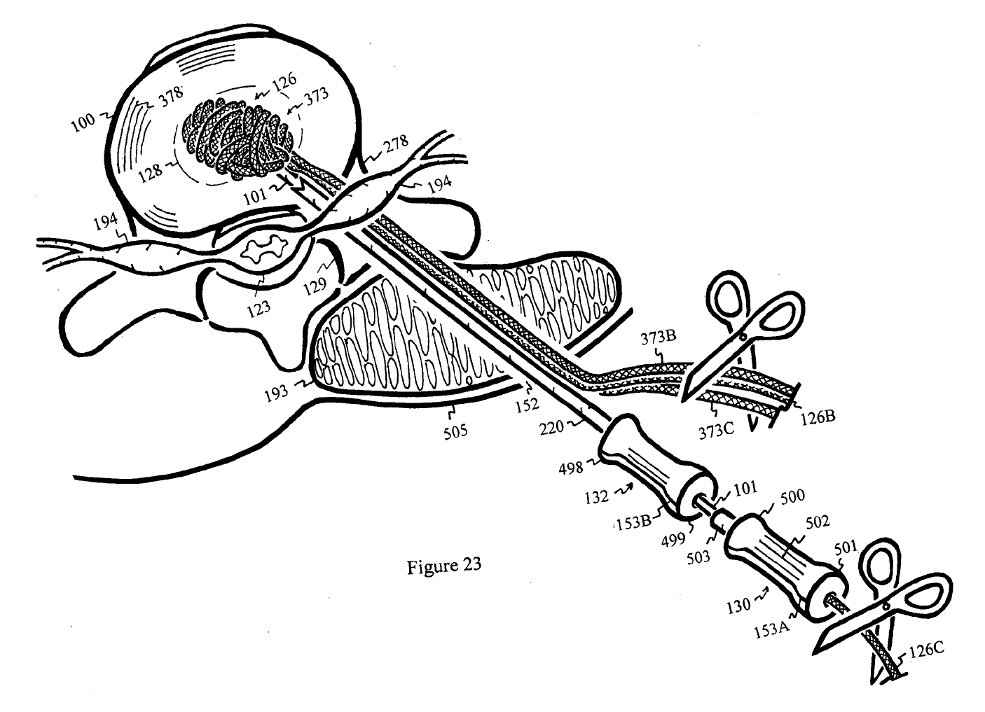

The portion of the shunt strand 126C at the lumen 269 opening can be excessively frail, weakened or partially torn from tension of spiraling in FIG. 15. A new portion of the shunt strand 126C is exposed by slightly withdrawing the needle 101 while holding the sleeve 220 stationary as shown in FIG. 20, then re-advancing the needle 101, so the Quincke tip 310 is even with the snagging points 221, similar to FIG. 19. The sleeve 220 is withdrawn while holding the needle 101 stationary, as shown in FIG. 21. The needle 101 is twisted or rotated again to spiral additional shunt strands or section 126B, 373B, 373C over the distal shaft of the needle 101, as shown in FIG. 22. In the event that tension of winding shunt strands 126B, 373B, 373C is not felt during needle 101 twisting, the shunt strand 126C extending from proximal end of the needle handle 130, as shown in FIG. 14, is pulled to re-establish contact between the U-section 126A and the beveled tip of the needle 101 for catching and spiraling the U-section 126A over the beveled tip of the needle 101. If location of the Quincke tip 310 is still in the nucleus 128, a slight advancement of the needle 101 also helps to re-engage the U-section 126A with the beveled tip of the needle 101 for additional spiraling of shunt strands or section 126B, 373B, 373C. Positions of the shunt strand 126C in the needle 101, the U-section 126A at the lumen opening 269 and strand 126B outside allow for re-adjustments and repetitive spiraling and deployment of strands 126B, 126C, 373B, 373C into the disc 100. The linked shunt strand 373 can be optional, but it adds bulk, size, mass and fluid transport, especially as external disc shunts 126, 373.

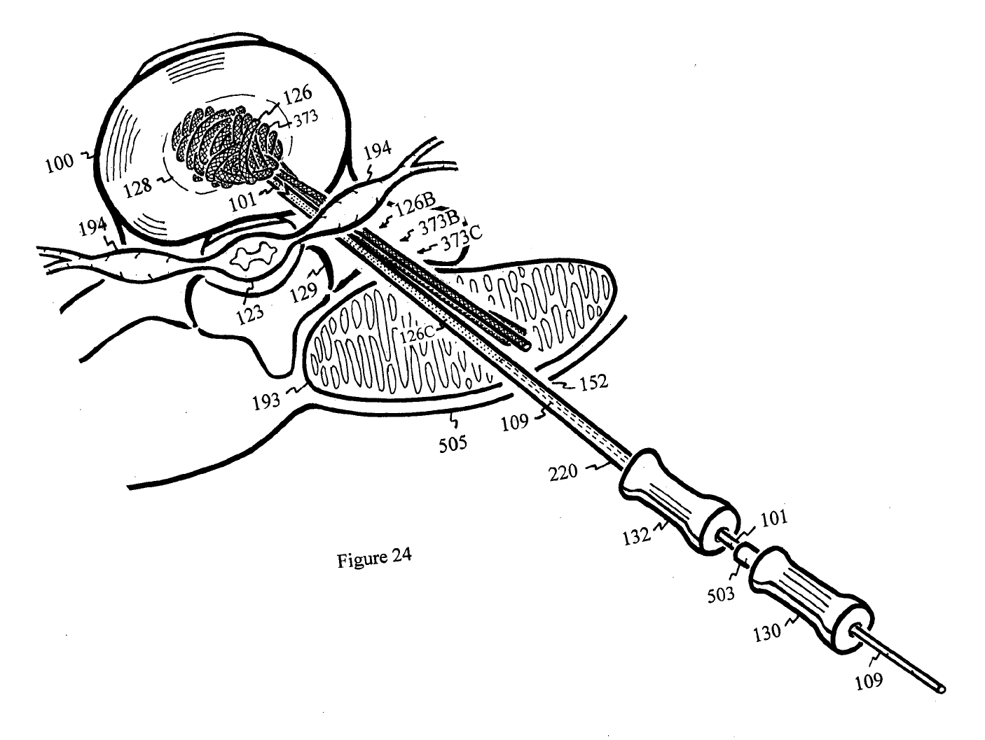

Additional spiraled or coiled shunt strands or sections 126B, 373B, 373C are delivered or dislodged individually, packing into the disc 100 by advancement of the sleeve 220 to fill the weak, malleable, flabby or sponge-like area or vacuole 184, within the degenerated disc 100. When the disc 100 is nearly full, packing of coiled or spiraled shunt strands 126B, 126C, 373B, 373C becomes more difficult, requiring more force to push the sleeve 220. The outside shunt strands 126B, 373B, 373C are cut above the skin 505, and the shunt strand 126C extending from the proximal opening of the needle handle 130 is also cut, as shown in FIG. 23. Additional shunt spiraling by the needle 101 and dislodgement by the sleeve 220 draw, reel or pull the shunt strands 126B, 373B, 373C under the skin 505 and within the muscle 193, as shown in FIG. 24.

The follow steps advance the shunt strand 126C within the lumen 269 of the needle 101 under the skin 505. Starting from the position of the shunt delivering device depicted in FIG. 21: (1) Rotate the needle 101 about twice, which winds only the shunt strand 126C over the needle 101 shaft. (2) Advance the sleeve 220 to dislodge the spiraled shunt strand 126C into the coils of spiraled shunt strands 126, 373, as shown in FIG. 24. (3) Withdraw the needle 101 about 1 cm, then re-insert the needle 101 for about 1 cm to position additional shunt strand 126C in the disc 100. (4) Withdraw the sleeve 220 to the needle handle 130. (5) Detect depth of the strand 126C within the needle 101 by inserting a dip stick 109 into the lumen 269 of the needle 101 through a proximal opening of the needle handle 130 as shown in FIG. 24. If the end of strand 126C is not beneath the skin 505, repeat the steps (1) to (5), until strands 126C, 126B, 373B, 373C are beneath the skin 505 and in the muscle 193. (6) Withdraw the needle 101 and sleeve 220 from the skin 505 after forming the internal and external disc shunts 126, 373, as shown in FIG. 25

In essence, the needle 101 has two positions. First position of the needle 101 is with the shunt strands 126B, 373B, 373C draping or residing outside the needle 101. Second position of the needle 101 has the shunt strands 126B, 373B, 373C spiraling, coiling, wrapping or winding over the beveled needle 101 shaft; the spiraling, coiling or wrapping is preferred to be on the distal portion of the needle 101. The conversion between the first and second position of the needle 101 is achieved by twisting or rotating the needle 101 to spiral, coil, reel or wind the shunt strands 126B, 373B, 373C over the beveled tip 310 at the distal end of the needle 101.

The sleeve 220 and the snagging point 221 also have two positions when sliding longitudinally over the beveled needle 101. In position one, the distal snagging point 221 is located proximal to the Quincke tip 310 of the needle 101. In position two, the snagging point 220 is located at, near, substantially level or substantially even with the Quincke tip 310 of the needle 101. During sliding from the position one to the position two, the snagging point 221 of the sleeve 220 maintains a fixed distance to the needle 101 shaft or the needle 101 outer wall. In position two, the snagging point 221 catches and dislodges the spiraled shunt strands 126B, 373B, 373C from the needle 101.

In the second position of the needle 101 and position one of the sleeve 220, the spiraled shunt strands or sections of 126B, 373B, 373C are mostly distal to the snagging point 221. During traveling or sliding from the position one to the position two of the sleeve 220, the snagging point 221 dislodges the spiraled shunt strands or sections 126B, 373B, 373C from the distal portion of the needle 101 into the disc 100, to convert from the second to the first position of the needle 101.

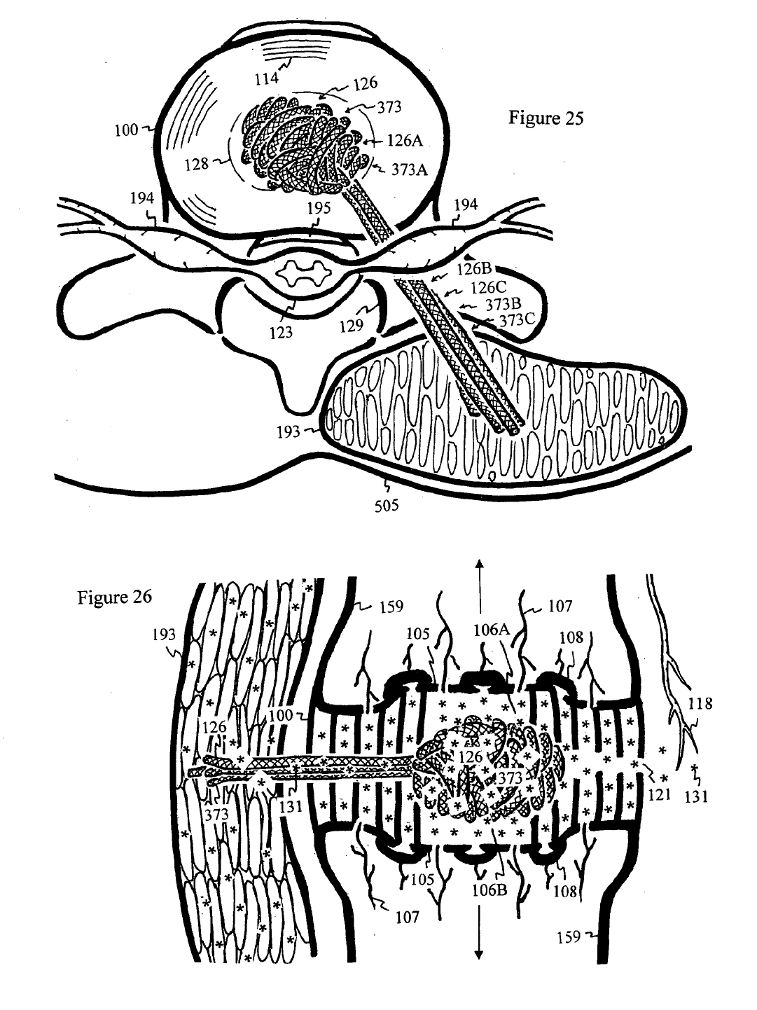

FIG. 26 shows a longitudinal view of a shunted disc 100 with calcified layers 108 accumulated over the endplate 105. The spiraled, coiled or knotted disc shunts 126, 373 reach, locate, reside or contact at least one of the superior 106A and inferior 106B diffusion zones, drawing and transporting nutrients/oxygen/pH buffer 131 to neutralize lactic acid 162 and nourish cells in the mid layer of the disc 100. The spiraled, coiled or knotted shunt strands are the internal disc shunts 126, 373 which relieve discogenic pain from lactic acid 162 burn. Bicarbonate and other pH buffering solutes 131 in the superior 106A and inferior 106B diffusion zones are absorbed, drawn and stored by the spiraled shunts 126, 373. Due to compression and relaxation of the disc 100 from daily activities of the patient, bicarbonate and other pH buffering solutes 131 are released or squeezed from the spiraled internal disc shunts 126, 373 in the lactic acid zone or mid layer of the disc 100 to neutralize the lactic acid 162. In essence, the internal disc shunts 126, 373 expand the superior 106A and inferior 106B diffusion zones, covering, erasing, inundating or obliterating the lactic acid zone in the central-mid layer of the disc 100. Hence, fluid leaking from the fissure 121 is pH neutral or near pH neutral to alleviate or reduce pain, as shown in FIG. 26.

The shunt strands 126B, 126C, 373B, 373C can also extend from the spiraled or coiled internal disc shunts 126, 373 within the disc 100 to muscle 193 or bodily circulation to draw nutrient/oxygen/pH buffer 131 into the disc 100, as external disc shunts 126, 373, shown in FIGS. 25-27.

Fluid flows from low to high osmolarity. External disc shunts 126, 373 were implanted into sheep (430 mOsm/liter) and human cadaver discs (300-400 mOsm/liter) of various degenerative levels, Thompson Grade 0-4. The shunted specimens were submerged in saline with blue dye (350 mOsm/liter). Dissection of the specimens showed blue saline permeation into the nuclei of all externally shunted discs.

Another external disc shunt 126, 373 was implanted through a muscle into a sheep disc. The sheep muscle was saturated with iopamidol (contrast agent with blue dye, 545 mOsm/l). The blue iopamidol did not permeate through the external shunt 126, 373 into the sheep disc (430 mOsm/liter). In fact the dissected disc looked desiccated; fluid within the sheep disc was probably drawn into the muscle infused with 545 mOsm/liter blue iopamidol through the external disc shunt 126, 373. The experiment was repeated with diluted blue iopamidol solution (150 mOsm/liter). The diluted iopamidol solution saturated the muscle and permeated through the external disc shunt 126, 373 into the sheep disc visible and traceable from muscle to nucleus under CT. Dissection confirmed permeation of the diluted blue iopamidol into the nucleus of the sheep disc.

More external disc shunts 126, 373 were implanted into sheep discs, then submerged in pork blood (about 300 mOsm/liter). Dissection of the specimens showed pork blood permeation through the external disc shunts into the gelatinous nuclei of the sheep discs (430 mOsm/liter).

In-vivo sheep study, implanted internal and external disc shunts 126, 373 showed no tissue reaction within the discs 100 or tissues adjacent to the discs 100 after 1, 3, 6 and 12 months study with histology staining. Color photo of the histology is shown in the U.S. Provisional Application 61/399,088, Alleviate back pain by expanding the diffusion zones, filed on Jul. 6, 2010. In addition, no adverse reaction occurred to the external disc shunts 126, 373 in human during a pilot study.

Osmolarity of human blood is about 300 mOsm/liter. Evidence indicates that nutrients/oxygen/pH buffer 131 in blood plasma of the muscle 193 and/or capillaries 107 at the endplate 105 flow through the hydrophilic or fluid absorbing internal and/or external disc shunt 126, 373 into the desiccated disc 100 with high osmolarity.

Furthermore, oxygen 131 from the superior 106A, and inferior 106B diffusion zones and muscle 193 converts anaerobic into aerobic conditions within the central-mid layer of the disc 100. Hence, in the presence of oxygen 131, production of lactic acid 162 may decrease significantly to further reduce lactic acid burn.

Compression and relaxation of the disc 100 from patient's daily activities behave similar to a diaphragm pump, drawing fluid from the diffusion zones 106A, 106B, and/or muscle 193 through the shunts 126, 373 into the mid layer of the disc 100, then expelling the fluid through the fissure 121. Fluid flow in the internal and/or external shunted disc 100 becomes dynamic, nutrients/oxygen/pH buffer 131 are re-supplied or replenished through the superior 106A and/or inferior 106B diffusion zones and/or muscle 193.

The multiple coiled or spiraled disc shunts 126, 373 provide bulk, shimming, filling, cushion, mass, wedging or fortification within the disc 100 to elevate, raise, lift, increase or sustain disc 100 height as indicated by arrows in FIG. 26. The spiraled disc shunts 126, 373 also serve as a filler or stabilizer to support and repair the flabby disc 100 from within. The repaired disc 100 in FIG. 27 becomes firm, stiff and/or thickened to reduce spinal instability. Disc height increases or elevates; difference can be compared or measured before and after implantation of spiraled disc shunts 126, 373 using standing X-rays. During compressive loading on the spine, the load is shifted from the inferior articular process 143 to the shunted disc 100, as shown in FIG. 27. Hence, the compressive load, strain and pain of the facet joints 129 are reduced.

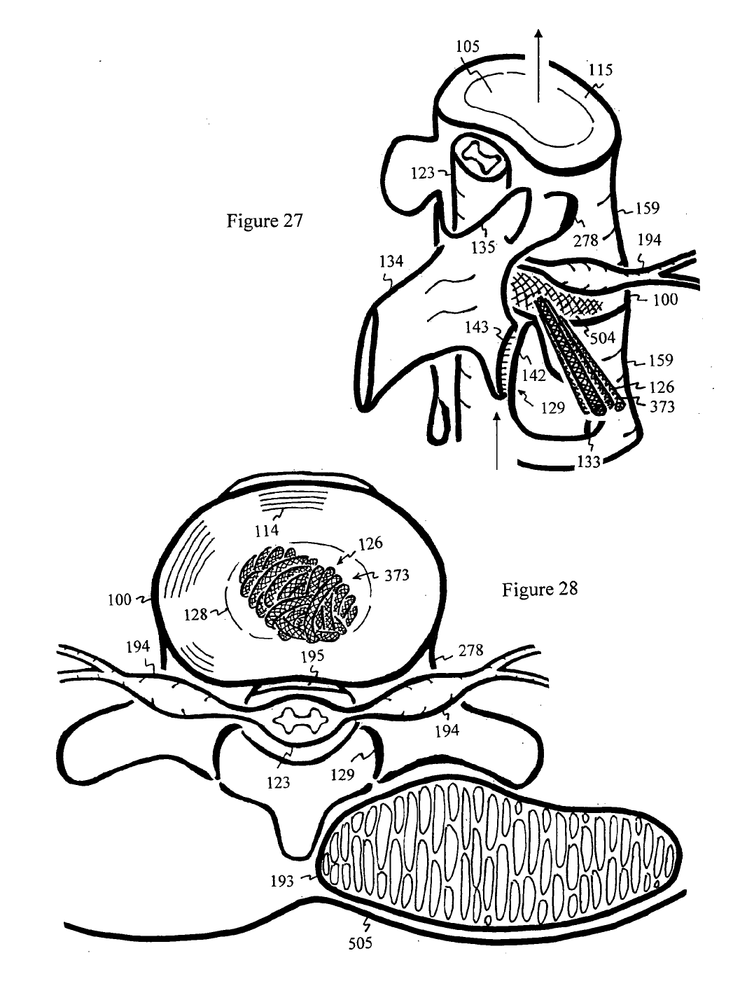

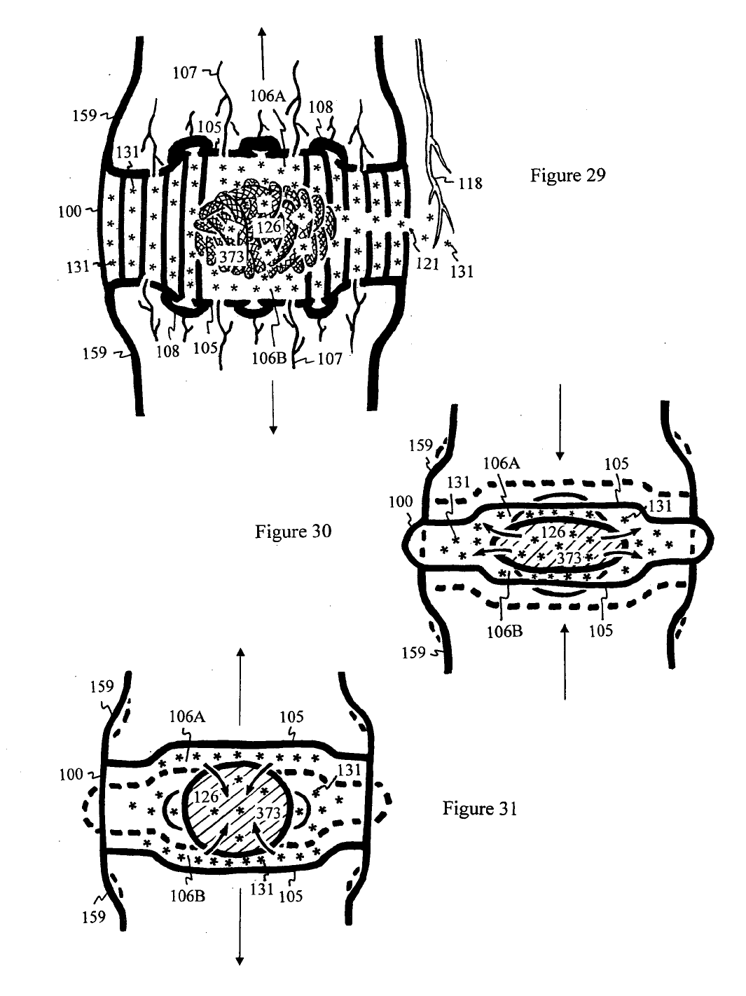

Nutrients 131 are diffused from the capillaries 107 at the endplates 105 into the nutrient-poor avascular disc 100, as shown in FIG. 26. Diffusion is concentration related; solutes moves from high to low concentration, from capillaries 107 into diffusion zones 106A, 106B. Due to drawing of nutrients 131 into the internal disc shunts 126, 373, concentration of nutrients 131 at the superior 106A and/or inferior 106B diffusion zones is reduced. Additional diffusion of nutrients 131 will be re-supplied through the capillaries 107 vascular buds. The net supply of nutrients/oxygen/pH buffer solutes 131 into the disc 100 will increase with implantation of the internal shunt 126, 373, as shown in FIGS. 28 and 29. The concentration gradient of nutrients/oxygen/pH buffer solutes 131 is extended or expanded by the internal shunts 126, 373, covering, diffusing or permeating the full-thickness of the intervertebral disc 100 to neutralize lactic acid 162, nourish starving disc cells 277 and rebuild disc matrix to sustain compressive loading of the spine.

FIG. 28 shows the internal disc shunts 126, 373 entirely spiraled, coiled, knotted or deployed within the disc 100, to increase supply of nutrients/oxygen/pH buffer 131 especially into the mid layer of the disc 100. FIG. 29 shows that the internal shunts 126, 373 reach, locate, absorb and/or draw nutrients 131 from at least one of the superior 106A and inferior 106B diffusion zones into the mid layers of the disc 100, expanding the diffusion zones and extending concentration gradient of the nutrient 131 into the central mid layer of human disc 100.

Depending on severity of the calcified layers 108 covering the capillaries 107 and vascular buds at the endplates 105, the superior 106A and inferior 106B diffusion zone containing nutrients/oxygen/pH buffer 131 are between 1 and 5 mm from the cartilaginous endplates 105. For degenerated and/or painful discs 100, the superior 106A and inferior 106B diffusion zones are likely between 0 and 3 mm from the superior and inferior endplates 105. Hence, the internal disc shunts 126, 373 should reach at least one, but preferably both superior 106A and inferior 106B diffusion zones, between 0 and 3 mm from both endplates. Repetitive formations and deployments of the coiled or spiraled shunt strands 126A, 126B, 126C, 373A, 373B, 373C are used to position, reside, locate, reach or contact at least one diffusion zones 106A, 106B, between 0 and 3 mm from at least one endplates 105 to form the internal disc shunt 126, 373. Distance of the internal disc shunt 126, 373 from the endplate 105 determines availability or quantity of nutrients/oxygen/pH buffer 131 for supplying the mid layer of the disc 100 to alleviate discogenic pain from lactic acid 162 burn.

In summary, insertion of the internal disc shunt 126, 373 increases the depth of diffusion of nutrients/oxygen/pH buffer 131 to neutralize lactic acid 162 and nourish disc cells in the mid layer of the disc 100. Furthermore, the internal disc shunts 126, 373 also add bulk, cushion, filling, thickness or fortification, as depicted by arrows in FIG. 29, to reduce or alleviate pain from the facet joints 129 and spinal instability, in FIG. 27.

The disc shunt strands 126, 373 are hydrophilic with measurable characteristics under ambient temperature and pressure for transporting and retaining fluid to relieve pain and/or regenerate the degenerated disc 100. After saturation in water, the disc shunts 126, 373 gain weight between 10% and 500% by absorbing water within the matrix of the disc shunt strands 126, 373. A healthy human disc 100 contains 80% water. The preferred water absorbency after water saturation is between 30% and 120%. The shunt strands 126, 373 can have pore sizes between 1 nano-meter and 200 micro-meters, serving as water retaining pockets or water transporting channels. Pores 124 of the disc shunt strands 126, 373 also function as scaffolding or housing for cell 277 attachment and cellular proliferation. Water contact angle on the disc shunt strands 126, 373 is between 0 and 60 degrees. The preferred water contact angle of the shunt strands 126, 373 is between 0 and 30 degrees. Height of capillary action for drawing saline up the disc shunt strands 126, 373 is between 0.5 and 120 cm. The preferred height of capillary action of drawing saline is between 1 and 60 cm. Height of capillary action for drawing pork blood up the disc shunt strands 126, 373 is between 0.5 and 50 cm. The preferred height of capillary action for drawing pork blood up the disc shunt strands 126, 373 is between 1 cm and 25 cm. Saline siphoning transport rate through the disc shunt strands 126, 373 is between 0.1 and 10 cc per 8 hours in a humidity chamber. Human lumbar disc 100 loses between about 0.5 and 1.5 cc fluid per day due to compression. The saline siphoning transport rate through the disc shunt strands 126, 373 is preferred between 0.5 and 5 cc per 8 hours in a humidity chamber. Pork blood siphoning transport rate through the disc shunt strands 126, 373 is between 0.1 and 10 cc per 8 hours in a humidity chamber. The pork blood siphoning transport rate through the disc shunt strands 126, 373 is preferred between 0.5 and 3 cc per 8 hours in a humidity chamber.

The shunt strands 126, 373 used in the sheep and human clinical studies have the following physical properties under ambient temperature and pressure: (1) weight gain 80% after water saturation, (2) water contact angle zero degree, (3) height of capillary action 11 cm with pork blood, 40 cm with saline with blue dye, and (4) rate of siphoning pork blood 1.656+/−0.013 cc per 8 hours in a humidity chamber.

Average lactic acid concentration in painful lumbar disc 100 is about 14.5 mM, 15 cc or less in volume (Diamant B, Karlsson J, Nachemson A: Correlation between lactate levels and pH of patients with lumbar rizopathies. Experientia, 24, 1195-1196, 1968). An in-vitro study was conducted to show instant lactic acid neutralization by blood plasma. The spiraled shunt strands 126, 373 were formed within, and then extracted from a fresh portion of beef. Blood plasma absorbed in the spiraled shunt strands 126, 373 instantly neutralized 42% of the 14.5 mM, 15 cc of lactic acid solution, measurable by a pH meter.

Approximately 85% back pain patients show no nerve impingement under MRI or CT. A patient without nerve impingement suffered chronic back pain with visual analog score 9 out of 10 (most severe), and leg pain with visual analog score 8. Five days after implantation of the disc shunts 126, 373, the visual analog score dropped to 2.5 for her back pain, but the visual analog score persisted at 8 for leg pain. During 5.5-month follow-up, the visual analog score dropped to 2.0 for her back pain, and visual analog score dropped from 8 to zero for leg pain. Quick back pain relief may be contributed to instant lactic acid 162 neutralization by blood plasma of the patient to relieve acid burning of the adjacent sensory nerves 118. Leg pain may be caused by acid scaring of the spinal nerve 194 and chemical radiculitis, which takes time to relieve the pain.

The internal disc shunt 126, 373 is a fluid-transferring or delivery device, inserted into the nucleus 128 of a degenerated disc 100. The multiple coiled or spiraled internal disc shunts 126, 373 are shape-conforming, malleable, resilient or squeezable between endplates 105, as shown in FIG. 30. During compressive loading of the disc 100, nutrients/oxygen/pH buffer 131 absorbed in the shunts 126, 373 are squeezed out, and distributed throughout the disc 100. During relaxation of the disc 100, the spiraled internal disc shunts 126, 373 expand, absorb and draw nutrients/oxygen/pH buffer 131 from superior 106A and/or inferior 106B diffusion zones into the matrix of the shunts 126, 373, as shown in FIG. 31. Repetitive compression and relaxation cycles help to distribute and circulate nutrients/oxygen/pH buffer 131 within the disc 100. Distribution of nutrients 131 is made possible by the sponge-like internal disc shunt 126, 373 with hydrophilic and malleable properties, absorbing and delivering nutrients/oxygen/pH buffer 131 within the avascular disc 100.

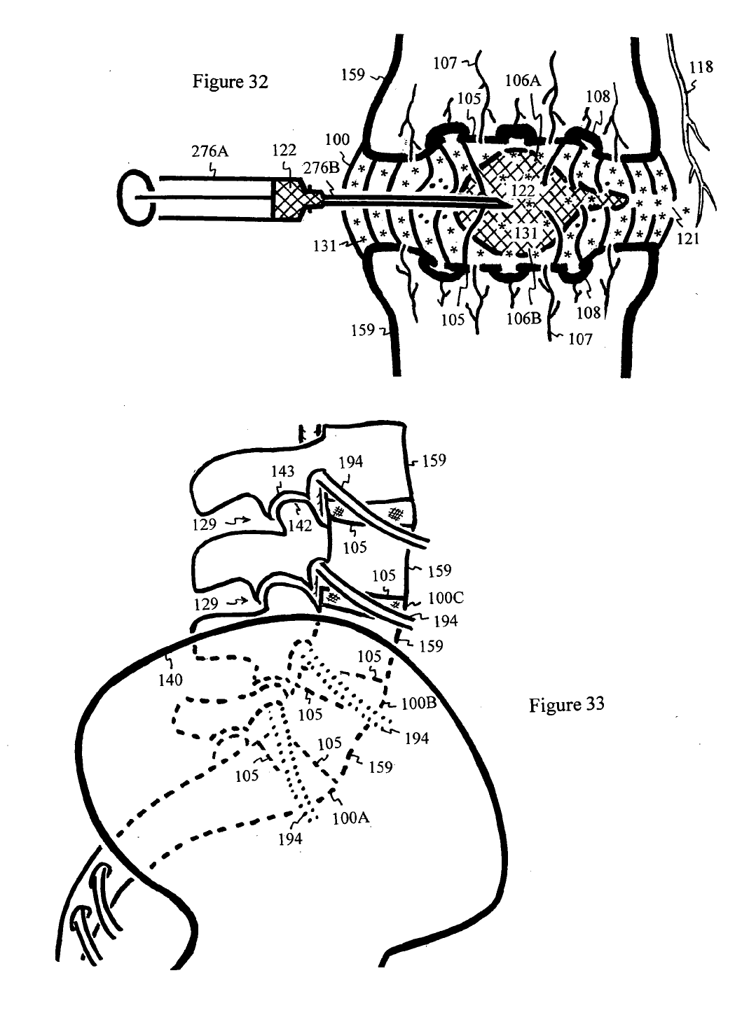

FIG. 32 shows injection of a hydrophilic gel, foam, viscous liquid or flowable liquid 122 into a disc 100. The injected gel, foam, viscous liquid or flowable liquid 122 is located in at least one of the superior 106A and inferior 106B diffusion zones. The superior 106A and inferior 106B diffusion zones are defined as depth into the disc 100, between 0 and 3 mm from the superior and inferior endplates 105 respectively. The injected gel, foam, viscous liquid or flowable liquid 122 is capable of drawing nutrients 131 from the superior 106A and inferior 106B diffusion zones into the mid layers of the disc 100. The hydrophilic gel, foam, viscous liquid or flowable liquid 122 is preferred having a shape changing or volume changing capability or characteristic, such as contraction and expansion for expelling and absorbing fluid, similar to a sponge. FIGS. 30 and 31 depict the shape or volume changing capability of an internal disc shunt 126, 373 during compression and relaxation of the spinal segment from daily activities of the patient, to help distributing nutrients/oxygen/pH buffer through out the degenerated disc 100. The hydrophilic gel, foam, viscous liquid or flowable liquid 122 has water contact angle between 0 and 60 degree in ambient temperature and pressure. The preferred water contact angle of the internal foam shunt 122 is between 0 and 30 degrees. After saturation in water, the hydrophilic gel, foam, viscous liquid 122 has water content between 10% and 700% under ambient temperature and pressure. The injectable gel, foam, viscous liquid or flowable liquid 122 becomes an internal foam shunt 122 to transport nutrients/oxygen/pH buffer from at least one of the superior 106A and inferior 106B diffusion zones into the mid layers of the disc 100 to neutralize the lactic acid 162 and nourish the disc cells.

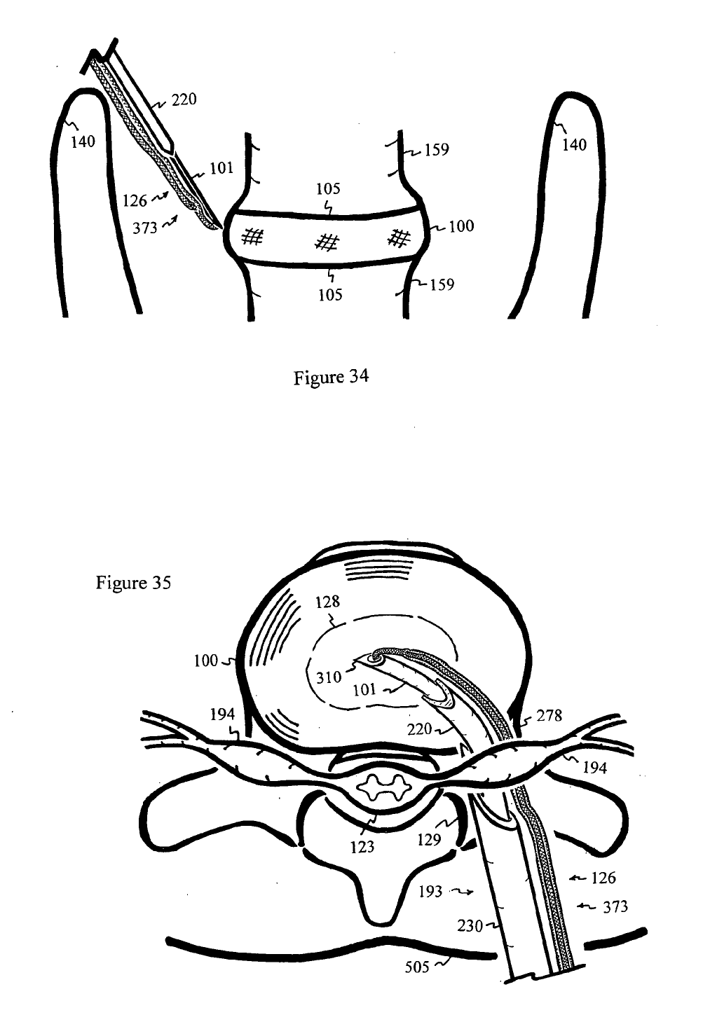

Lower lumbar L5-S1 disc 100A and L4-5 disc 100B are shielded by a pair of ilia 140, as shown in FIG. 33. The straight shunt delivery needle 101 enters superiorly over the ilium 140 at an angle, as shown in FIG. 34, difficult or even impossible to deliver the disc shunt strands 126, 373 into the nucleus 128 of the disc 100.

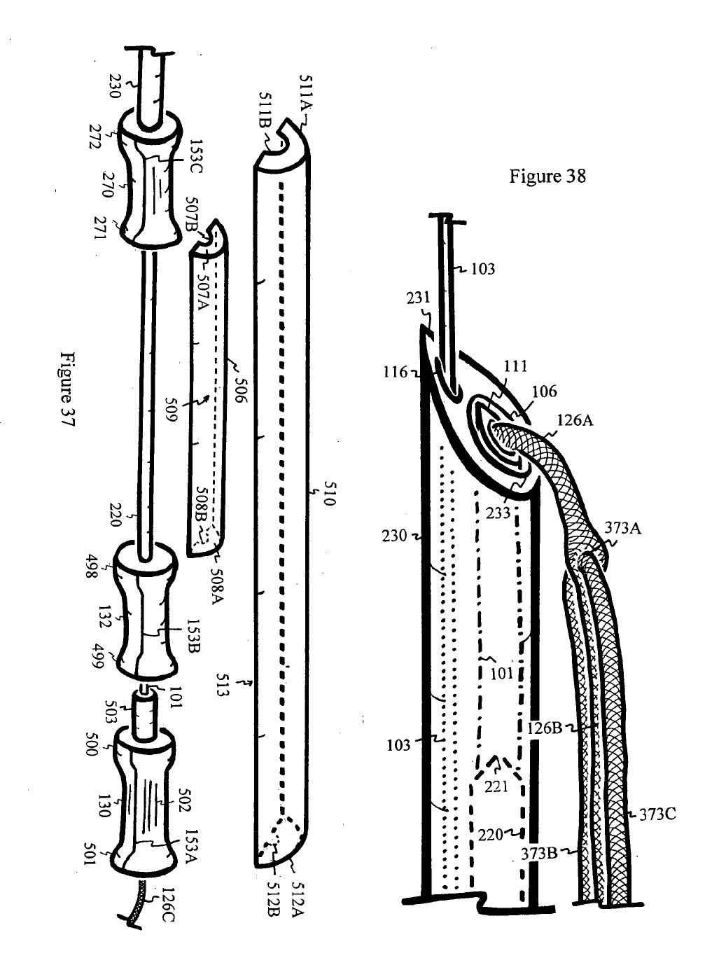

FIG. 35 shows a straight and rigid cannula needle 230, guided by fluoroscopy to the Kambin's Triangle 504 of a degenerated disc 100. Quincke sharp tip 231 of the cannula needle 230 is preferred facing and/or close to the facet joint 129 to avoid nicking the spinal nerve 194. An elastically curved needle 101 and sleeve 220 are resiliently straightened within the rigid cannula needle 230, as shown in FIG. 38. During fluoroscopic-guided deployment of the elastically curved needle 101 from the straight and rigid cannula needle 230, a sharp tip 310 located at the concave side of the curved needle 101 helps to steer the needle 101 into the nucleus 128 of the intervertebral disc 100. As steering spearhead, the sharp tip 310 at the concave side may reduce curvature of the shunt delivery needle 101 and sleeve 220, resulting in less strain in resiliently straightened positions within the rigid cannula needle 230.

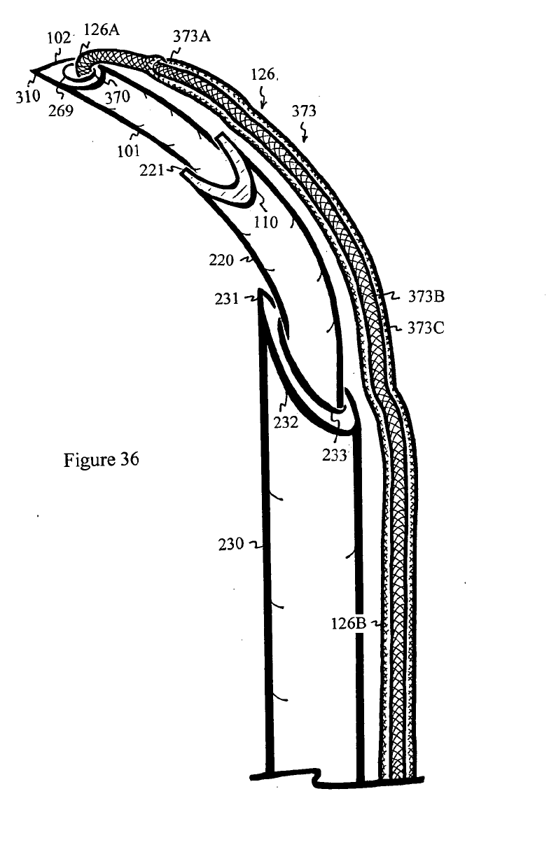

Similar to the shunt delivery needle 101, the cannula needle 230 has the sharp Quincke tip 231 with a dull distal external edge 232, shown in FIG. 36, for puncturing tissue and pushing nerves or blood vessels aside during body puncturing with the cannula needle 230. The shunt strands 126B, 373B and 373C drape along the outside wall of the cannula needle 230 to minimize size of the cannula needle 230, risk of injuring spinal nerve 194 and patient discomfort. The shunt strands 126B, 373B and 373C are press-fitted into the body of the patient, outside the outer wall of the cannula needle 230.

A handle 270 of the cannula needle 230 in FIG. 37 has a marker 153C showing orientation of the Quincke sharp tip 231, a distal protrusion 272 to facilitate cannula 230 advancement, and a proximal protrusion 271 to facilitate cannula 230 withdrawal.

Stacking of the needle 101, sleeve 220 and cannula 230 needs spacers to keep them apart and a holder 510 to keep the stack together, especially during tissue puncturing. A sleeve-cannula spacer 506 is required to keep the needle 101 and sleeve 220 from deploying past the distal lumen 111 of the cannula needle 230. The removable sleeve-cannula spacer 506 contains a trough-like cavity 509, with a distal opening 507B and a proximal opening 508B to house the sleeve 220. The sleeve-cannula spacer 506 also contains a distal wall 507A abutting the proximal protrusion 271 and a proximal wall 508A abutting the distal protrusion 498 of the sleeve handle 132. A removable tri-handle holder 510 contains a trough-like cavity 513 to house the cannula handle 270, sleeve-cannula spacer 506, sleeve handle 132, sleeve-needle spacer 503 and needle handle 130. The tri-handle holder 510 also contains a distal wall 511A to support the distal protrusion 272 of the cannula handle 270, and a proximal wall 512A to support the proximal protrusion 501 of the needle handle 130. The distal wall 511A contains an opening 511B, sized and configured to arch over the cannula 230. The proximal wall 512A contains another opening 512B, sized and configured to arch over the shunt strand 126C, as shown in FIG. 37. The tri-handle holder 510 unifies and fastens the cannula handle 270, sleeve-cannula spacer 506, sleeve handle 132, sleeve-needle spacer 503 and needle handle 130. A removable tie or band can be used to fasten, secure or bundle the tri-handle holder 510 with the handles 270, 132, 130 and sleeve-cannula spacer 506.

To improve accuracy and decrease procedural time, the cannula needle 230 can be guided by a guide wire 103 into the disc 100. Discography is often used to confirm discogenic pain using contrast 163 injection, as shown in FIG. 5. Aiming and positioning the needle 276B for discography takes time and skill. After confirming the discogenic pain, the syringe 276A for discography is removed, while the discography needle 276B remains. The guide wire 103 with blunted distal and proximal ends is inserted through the discography needle 276B into the disc 100. The proximal end of the guide wire 103 is held stationary during withdrawal of the discography needle 276B from the patient. The guide-wire lumen 116 of the cannula needle 230 is inserted over the proximal end of the long guide wire 103, as shown in FIG. 38. The proximal end of the guide wire 103 is held stationary during advancement of the cannula needle 230 toward the Kambin's Triangle 504. The main lumen 111 of the cannula 230 houses the resiliently straightened needle 101, sleeve 220 and shunt strand 126C. The U-section 126A is positioned near the distal lumen 111 opening of the cannula 230. The main and linked shunt strands 126B, 373A, 373B, 373C drape, dangle, reside, position or lay along the outside wall of the cannula needle 230, as shown in FIG. 38.

The guide wire 103 can also be inserted into the lumen 269 of the needle 101 with the shunt strand 126C, or into a separate longitudinal chamber or opening parallel with the lumen 269, for housing the guide wire 103 to facilitate needle 101 entry into the disc 100.

The tri-handle holder 510 and sleeve-cannula spacer 506 are removed when the proximal end of the guide wire 103 extends beyond the proximal protrusion 271 of the cannula handle 270. To avoid kinking the guide wire 103 during advancement of the cannula 230, the proximal portion of the guide wire 103 is held firmly while the cannula needle 230 is advanced into the body of the patient, toward the Kambin's Triangle 504 under fluoroscopic guidance. Needle positioning takes multiple X-rays, skill and time. Placement of the guide wire 103 allows the physician to diagnose then treat the pain by aiming or positioning the needle only once, as shown in FIGS. 5 and 38.

As mentioned, discography is a diagnostic technique for detecting or confirming discogenic pain by flushing lactic acid 162 to sensory nerves 118. Saline or other non-buffering solution can also be injected into, then aspirated from the disc 100, which may contain lactic acid 162. Acidity of the aspirated solution is checked with a pH electrode. If the aspirated solution is highly acidic, shunt strands 126. 373 with buffering or alkaline coating may be needed for instant pain relief.

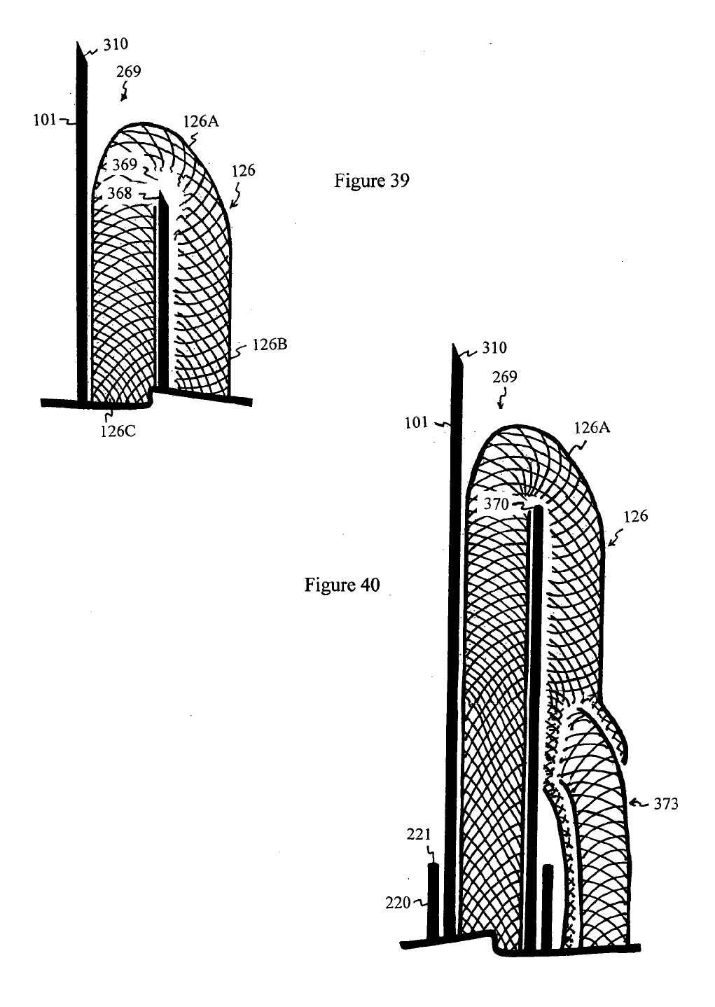

Needle 101 sharpening inevitably creates a semi-circular blade-like inner wall 368 at lumen opening 269, as shown in a mid-longitudinal view in FIG. 39. During in-vitro and in-vivo disc 100 puncturing to press-fit the U-section 126A of the shunt 126 into sheep discs 100, the blade-like inner wall 368 often sheared and damaged the U-section 126A. The damaged portion 369 of the U-section 126A forms small fibers or shedding debris 369 which can cause tissue reaction to the otherwise inert material. In fact, shearing was so serious that many U-sections 126A were severed during press-fit disc 100 puncturing.

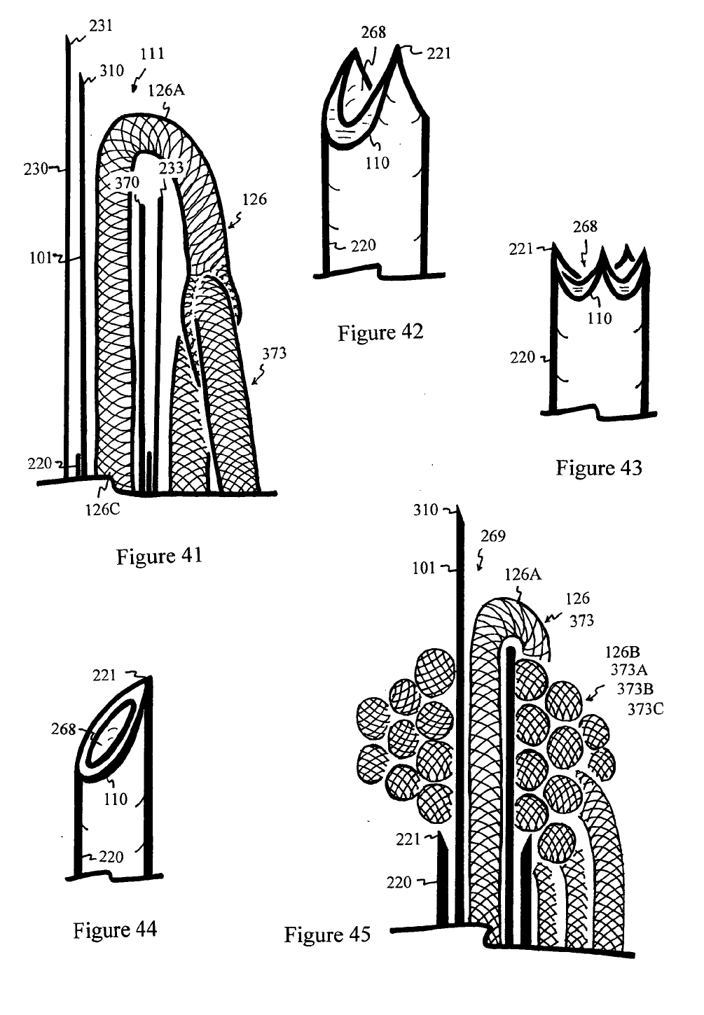

FIG. 40 shows a rounded or blunt inner wall or inner lip 370 at the lumen 269 opening of a needle 101. The rounded or blunt inner wall 370 can be formed by machining or filing to prevent damage to the U-section 126A during press-fit puncturing into the disc 100 or needle 101 rotation for spiraling shunt strands 126B, 373B, 373C. It is also possible to pad, cover, coat or fortify the U-section 126A to minimize damage by the sharp inner wall 368 of the needle 101. Similarly, a rounded or dull semi-circular inner wall 233 or inner lip is made at the lumen 111 of the cannula needle 230, as shown in FIG. 41, to prevent cutting or damaging the U-section 126A during tissue puncturing.

FIG. 42 shows the distal end of the sleeve 220 with a lumen 268 for housing and sliding over the needle 101. Two snagging points or tips 221 of the sleeve 220 are made with bi-beveling 110 of the distal end of the sleeve 220. The snagging points or tips 221 are preferred to be sharp, for snagging, catching, hooking, engaging, pinning, nailing pushing or dislodging the spiraled shunt strands 126B, 373B, 373C from the distal shaft of the needle 101. FIG. 43 shows four snagging points 221; and FIG. 44 shows a single snagging point 221 by beveling or indenting 110 the distal end of the sleeve 220.

The snagging point 221 can also be a distal wall, rim or end of the sleeve 220. FIG. 45 shows a mid-longitudinal view of spiraled shunt strands 126B, 373A, 373B, 373C over the distal shaft of the needle 101. The snagging points 221 are made by beveling or shaving the inner wall at the distal lumen opening 268 of the sleeve 220 to snag, catch, engage or dislodge the spiraled shunt strands 126B, 373A, 373B, 373C from the distal shaft of the needle 101.

The snagging point 221 can also be a rim or edge of an outer wall of the sleeve 220. The edge or rim is formed by a simple 90 degree cut on the sleeve 220.

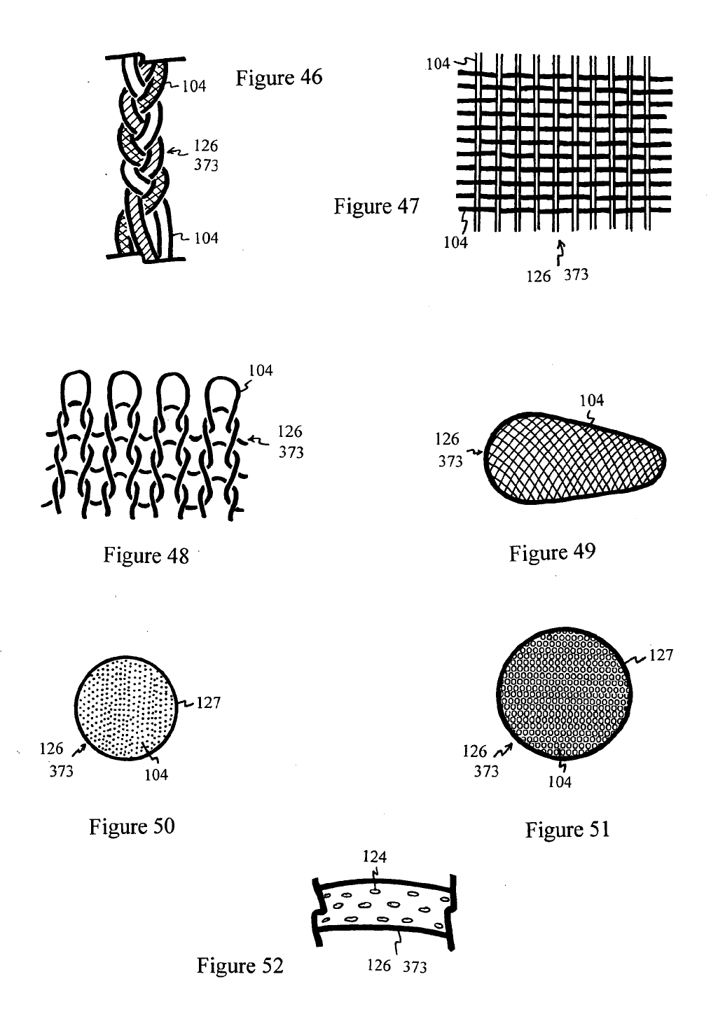

Flexible disc shunt strands 126, 373 can be made or formed by fabric making techniques, such as braiding or twisting filaments 104 as shown in FIG. 46. For twisting, minimum number of filaments 104 is two. For braiding, minimum number of filaments 104 is three, as shown in FIG. 46. Braiding is intertwining three or more filaments 104 for excellent flexibility, strength and porosity. The snagging point 221 can catch, snag or engage the spiraled braided shunt strands 126B, 373B, 373C well. The flexible disc shunt strands 126, 373 can also be woven, as shown in FIG. 47. Weaving is interlacing the filaments 104 over and under each other, generally oriented at 90 degree angles. Half of the filaments 104 from weaving can be oriented length-wise along the linear shunt strands 126, 373, to expedite fluid flow from the muscle 193 or diffusion zones 106A, 106B into the degenerated disc 100. The flexible disc shunt strands 126, 373 can be knitted, as shown in FIG. 48. Knitting is a construction made by interlocking loops of one or more filaments 104. A knitted shunt strands 126, 373 may have the greatest elasticity, capable of stretching and elongating during the press-fitted delivery into the disc 100. After the disc shunts 126, 373 are coiled, spiraled or reeled within the disc 100, diameters of the shunt strands 126B, 126C, 373B, 373C extending from the disc 100 expand, further sealing the needle tract to prevent the loss of hydrostatic pressure within the disc 100. In addition, the knitted shunts 126, 373 in coils, spirals or reels may have the highest porosity to enhance fluid absorbency, creating a reservoir of nutrients/oxygen/pH buffer 131 for dispersing into various parts of the avascular disc 100, as shown in FIGS. 30 and 31. Furthermore, the coiled or spiraled shunt strands 126, 373 with knitted filaments 104 provide an elastic cushion within the disc 100 to reduce loading and pain in the facet joints 129. The knitted shunt 126, 373 may be an excellent matrix or scaffolding for cell 277 attachment and proliferation. The disc shunt strand 126, 373 can be made with non-woven filaments 104. The term non-woven is used in fabric industry to include all other techniques, such as carded/needle-punched, spun bonded, melt blown or other. Non-woven disc shunts 126, 373 can provide large surface area as scaffolding for cell 277 growth and proliferation. Combinations of fabric making techniques can be used to form the internal and/or external disc shunts 126, 373. The main shunt 126 and the linked shunt 373 can be made with different material or different fabric making techniques. For example, the main shunt 126 can be made primarily for fluid transport, while the linked shunt 373 can be made primarily for cell 277 attachment and proliferation. The main shunt 126 and the linked shunt 373 can be coated with different substances to alleviate back pain and/or promote disc 100 regeneration.

Material and/or orientation of the filaments 104 of the disc shunts 126, 373 can affect (1) flow rate, (2) tensile strength, (3) annular sealing, (4) porosity, (5) fluid absorbency, (6) snagging ability, (7) elasticity, (8) selectivity of solute transport, (9) scaffold attachment of cells, (10) flexibility, (11) durability, (12) sterilization technique, (13) fibrotic formation, and/or (14) biocompatibility. A disc shunt 126, 373 is cut at a slanted angle, showing a cross-section of a shunt strand 126 or 373; the filaments 104 are slanted or diagonally oriented to the length-wise shunt strands 126, 373, as shown in FIG. 49. FIG. 50 shows cross-sections of filaments 104 parallel to the disc shunt strands 126, 373, covered by a wrapper, sheath or cover 127. The parallel-oriented filaments 104 and wrapper 127 can be manufactured by extrusion. The filaments 104 can also be micro tubes, as shown in FIG. 51, parallel to the disc shunt strands 126, 373. A wrapper 127 is used to cover, retain, enclose or house the micro tubular filaments 104 to form a strand of the disc shunts 126, 373. Individual micro tubular filament 104 is capable of having capillary action, drawing nutrients/oxygen/pH buffer 131 through the shunt strands 126, 373 into the disc 100.

The filaments 104 are preferred to be made with biocompatible and hydrophilic material, absorbing, retaining or drawing fluid with nutrients/oxygen/pH buffer solutes 131 from a tissue with low osmolarity to mid layer of the desiccated disc 100 with high osmolarity. The internal and/or external disc shunt strands 126, 373 can be a suture, approved for human implant. Instead of fastening tissue, the suture is used as disc shunts 126, 373, transporting fluid from low to high osmolarity to alleviate back pain.

The internal and/or external shunt strands 126, 373 can be made with a hydrophilic sponge or foam with pores 124, as shown in FIG. 52, to transport and retain fluid in the disc 100. The pores 124 can be open, connecting to other pores 124. The pores 124 can also be closed, not connecting to other pores 124 to retain fluid and cells 277.

Disc cells 277 isolated from advanced degenerated human discs 100 are still capable of producing collagen and glycosaminoglycans in tissue culture with abundant supply of nutrients in proper pH. (Gruber H. E., Leslie K., Ingram J., Hoelscher G., Norton H. J., Hanley E. N. Jr.: Colony formation and matrix production by human anulus cells: modulation in three-dimensional culture, Spine, July 1, 29(13), E267-274, 2004. Johnstone B, Bayliss M T: The large proteoglycans of the human intervertebral disc, Changes in their biosynthesis and structure with age, topography, and pathology, Spine, March 15; 20(6):674-84, 1995.) Furthermore, stem cells have recently been found in degenerated discs. (Risbud M V, Gattapalli A, Tsai T T, Lee J Y, Danielson K G, Vaccaro A G, Albert T J, Garzit Z, Garzit D, Shapiro Evidence for skeletal progenitor cells in the degenerate human intervertebral disc, Spine, November 1; 32(23), 2537-2544, 2007.) Nutrient 131 deficiency and acidic pH may hinder disc 100 repair in-vivo.

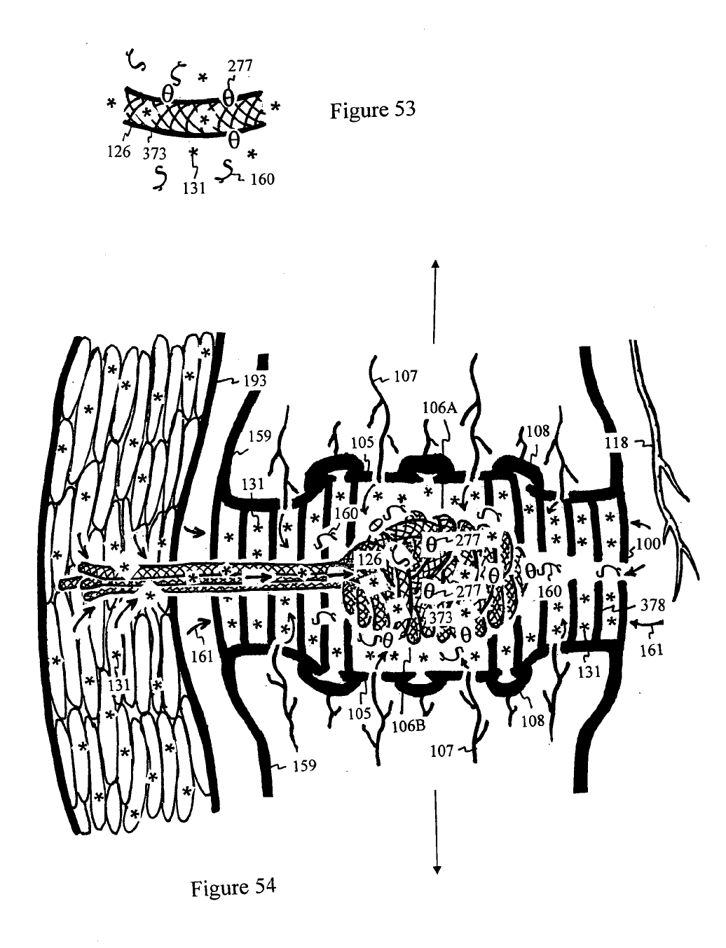

The internal and/or external disc shunts 126, 373 can be scaffolds and spigots for supplying nutrients/oxygen/pH buffering solute 131 for cells 277 to attach, as shown in FIG. 53. With a continual or renewable supply of nutrients/oxygen/pH buffer solutes 131, disc cells 277 resume making biosynthetic products 160, such as the water-retaining glycosaminoglycans and collagen, the major components of the nucleus 128 and annulus 378, as depicted in FIGS. 53-54. In sheep study, newly formed glycosaminoglycans can be seen on filaments 104 of the disc shunt 126, 373 after 3 months using Safranin histological staining.

The rate of sulfate incorporation for biosynthesizing glycosaminoglycans is pH sensitive. The maximum rate of sulfate incorporation is with pH 7.2-6.9. The rate of sulfate incorporation drops about 32-40% in acidic pH within the disc [Ohshima H, Urban J P: The effect of lactate and pH on proteoglycan and protein synthesis rates in the intervertebral disc. Spine, September: 17(9), 1079-82, 1992]. Hence, pH normalization with pH buffer solute 131 through the disc shunts 126, 373 will likely increase production of the water-retaining glycosaminoglycans and swelling pressure of the shunted disc 100.

With continual supply of nutrients 131, newly formed biosynthetic products 160 increase osmolarity within the disc 100 and enhance inward fluid flow 161, as shown in FIG. 54. The increased fluid flow 161 comes through (1) the internal and/or external disc shunts 126, 373, (2) blood capillaries 107 through the endplates 105, and/or (3) annulus 378. The fluid is also retained by the newly formed water-retaining glycosaminoglycans 160. As a result, swelling pressure of the shunted disc 100 increases. Segmental or spinal instability is reduced. Muscle tension and ache from guarding the spinal instability decrease. Load and pain of the facet joints 129 decrease. Lactic acid is further neutralized by inflow 161 of nutrients/oxygen/pH buffering solute 131 to reduce or alleviate acid burn. Disc 100 height is elevated, raised or increased as depicted by arrows in FIG. 54. In essence, implantation of the internal and/or external disc shunts 126, 373 enables the degenerated disc 100 to be repaired.

Furthermore, adenosine triphosphate, ATP, is the high-energy compound essential for driving or energizing biochemical reactions, including the biosynthesis of the water retaining glycosaminoglycans for sustaining compressive loads on the disc 100. Under anaerobic conditions, metabolism of each glucose molecule produces only two ATP and two lactic acids 162, which irritate adjacent nerves 118. When oxygen 131 permeates through the internal and/or external disc shunts 126, 373, thirty-six ATP can be produced from each glucose molecule through glycolysis, citric acid cycle and electron transport chain under aerobic conditions to energize disc regeneration and alleviate back pain.

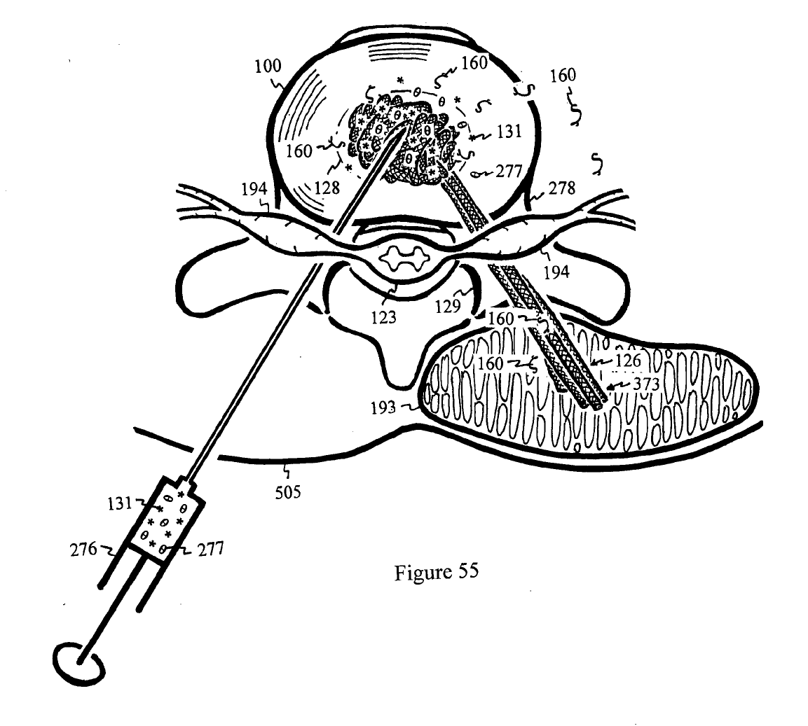

High concentration of nutrients 131 can also be injected into the internal and/or external shunted disc 100 to instantly create high osmolarity, as shown in FIG. 55. High osmolarity promotes fluid inflow 161 into the shunted disc 100. However, glucose or sugars injection can produce additional lactic acid 162, causing more pain. Sulfate and amino acids can be injected in high concentration to boost osmolarity and production of glycosaminoglycans and collagen, as the biosynthetic product 160 in FIG. 55. Magnesium, potassium, or sodium sulfate has high water solubility. Proline and glycine also have reasonably high water solubility and are essential nutrients 131 for biosynthesis of collagen in the annulus 378.

Analgesics, anti-depressant, steroid, NSAID, antibiotics, anti-inflammatory drugs, alkaline agent or other drugs can also be injected into the internal and/or external shunted disc 100 to further reduce pain.

Autograft disc cells 277 from a healthy disc 100 of the patient can be transplanted into the degenerated and shunted disc 100 to promote disc regeneration and production of biosynthetic product 160, as shown in FIG. 55.

The avascular disc 100 is well sealed. Even small ions, such as sulfate, and small molecules, such as proline, are greatly limited from diffusing into the nucleus pulposus 128. The well sealed disc 100 may be able to encapsulate donor cells 277 from a disc 100 of another person, cadaver or even animal without triggering an immune response. For disc 100 regeneration, the donor cells 277 can also be stem cells 277, notochord 277 or chondrocytes 277. The internal and/or external disc shunts 126, 373 are permeable to nutrients/oxygen/pH buffering solute 131 but impermeable to cells and/or cytokines responsible for triggering an immune reaction. The cells of the immune system include giant cells, macrophages, mononuclear phagocytes, T-cells, B-cells, lymphocytes, Null cells, K cells, NK cells and/or mask cells. The cytokines may also include immunoglobulins, IgM, IgD, IgG, IgE, other antibodies, interleukins, lymphokines, monokines or interferons.

The molecular weights of nutrients 131 and lactic acid 162 are much smaller than the immuno-responsive cells and cytokines. The transport selectivity can be regulated or limited by the size of the pores or channels within the semi-permeable internal and/or external shunts 126, 373. The upper molecular weight cut-off of the disc shunts 126, 373 can be 3000 or lower to allow the passage of nutrients and waste but exclude the immuno-responsive cells and cytokines. The semi-permeable disc shunts 126, 373 may also contain ionic or affinity surfaces to attract nutrients 131 and waste, including lactic acid 162. The surfaces of the semi-permeable disc shunts 126, 373 can be made, coated or modified to repel, exclude or reject immuno-responsive components.

In recent years, cell transplants from cadavers or live donors have been successful in providing therapeutic benefits. For example, islet cells from a donor pancreas are injected into a type I diabetic patient's portal vein, leading into the liver. The islets begin to function as they normally do in the pancreas by producing insulin to regulate blood sugar. However, to keep the donor cells alive, the diabetic patient requires a lifetime supply of anti-rejection medication, such as cyclosporin A. In addition to the cost of anti-rejection medication, the side effects of these immuno-suppressive drugs may include cancer. The benefit of cell transplant may not out weigh the potential side effects.

The intervertebral disc 100 with semi-permeable internal and external disc shunts 126, 373 can be used as a semi-permeable capsule to encapsulate the injected therapeutic donor cells 277 or agent, as shown in FIG. 55, to evade the immune response; hence no life-long immuno-suppressive drug would be required. A variety of donor cells 277 or agent can be harvested and/or cultured from the pituitary gland (anterior, intermediate lobe or posterior), hypothalamus, adrenal gland, adrenal medulla, fat cells, thyroid, parathyroid, pancreas, testes, ovary, pineal gland, adrenal cortex, liver, renal cortex, kidney, thalamus, parathyroid gland, ovary, corpus luteum, placenta, small intestine, skin cells, stem cells, gene therapy, tissue engineering, cell culture, other gland or tissue. The donor cells 277 are immunoisolated within the shunted discs 100, the largest avascular organs in the body, maintained by nutrients 131 and waste transport through the semi-permeable shunts 126, 373. The donor cells 277 can be from human, animal or cell culture. When disc pressure is low during sleep or supine position, nutrients/oxygen/pH buffering solutes 131 are supplied through the internal and external shunts 126, 373 to the donor cells 277. During waking hours while the pressure within the disc 100 is high, biosynthesized products 160 by these donor cells 277 are expelled through the shunts 126, 373 into the muscle 193, as shown in FIG. 55, or through fissures 121 into bodily circulation and target sites.

The biosynthesized product 160 made by the donor cells 277 nourished by the internal and external shunted disc 100 can be adrenaline, adrenocorticotropic hormone, aldosterone, androgens, angiotensinogen (angiotensin I and II), antidiuretic hormone, atrial-natriuretic peptide, calcitonin, calciferol, cholecalciferol, calcitriol, cholecystokinin, corticotropin-releasing hormone, cortisol, dehydroepiandrosterone, dopamine, endorphin, enkephalin, ergocalciferol, erythropoietin, follicle stimulating hormone, γ-aminobutyrate, gastrin, ghrelin, glucagon, glucocorticoids, gonadotropin-releasing hormone, growth hormone-releasing hormone, human chorionic gonadotrophin, human growth hormone, insulin, insulin-like growth factor, leptin, lipotropin, luteinizing hormone, melanocyte-stimulating hormone, melatonin, mineralocorticoids, neuropeptide Y, neurotransmitter, noradrenaline, oestrogens, oxytocin, parathyroid hormone, peptide, pregnenolone, progesterone, prolactin, pro-opiomelanocortin, PYY-336, renin, secretin, somatostatin, testosterone, thrombopoietin, thyroid-stimulating hormone, thyrotropin-releasing hormone, thyroxine, triiodothyronine, trophic hormone, serotonin, vasopressin, or other therapeutic products. These biosynthetic products 160 have low molecular weights and are able to be transported through disc shunts 126, 373 and/or fissures 121, while the donor cells 277 are trapped within the disc 100.

The biosynthesized products 160 (hormones, peptides, neurotransmitter, enzymes, catalysis or substrates) generated within the internal and/or external shunted disc 100 may be able to regulate bodily functions including blood pressure, energy, neuro-activity, metabolism, and activation and suppression of gland activities. Some hormones and enzymes govern, influence or control eating habits and utilization of fat or carbohydrates. These hormones or enzymes may provide weight loss or gain benefits. Producing neurotransmitters, such as dopamine, adrenaline, noradrenaline, serotonin or γ-aminobutyrate, from the donor cells 277 within the shunted disc 100 can treat depression, Parkinson's disease, learning disability, memory loss, attention deficit, behavioral problems, mental or neuro-related diseases.

Release of the biosynthesized products 160 by the donor cells 277 within the internal and/or external shunted disc 100 is synchronized with body activity. During activities of daily living, the pressure within the shunted disc 100 is mostly high to expel the biosynthesized products 160 by the donor cells 277 into circulation to meet the demands of the body. In the supine position, pressure within the shunted disc 100 is low; fluid inflow 161 through the internal and/or external shunts 126, 373 is favorable, bringing nutrients/oxygen/pH buffer 131 into the disc 100 to nourish the cells 277. As an example, islets of Langerhans from a donor's pancreas are implanted or injected into the shunted disc 100. In supine position during sleeping, glucose enters into the shunted disc 100 to induce production of insulin from the implanted islets of Langerhans. During waking hours when disc pressure is high, insulin is expelled through the shunts 126, 373 or fissure 121 into circulation to regulate concentration of glucose in the body. At night, the insulin released from the shunted disc 100 is minimal to prevent the hypoglycemia. In essence, biosynthesized products 160 by the donor cells 277 are released concurrent with physical activity to meet the demands of the body.

Donor cells 277 can also be seeded on the shunt strands 126, 373, or injected days, weeks, months or even years after implanting the internal and/or external disc shunts 126, 373, to ensure favorable biological conditions, including pH, electrolytic balance and nutrients and oxygen 131, for cell 277 survival and proliferation in the shunted disc 100.

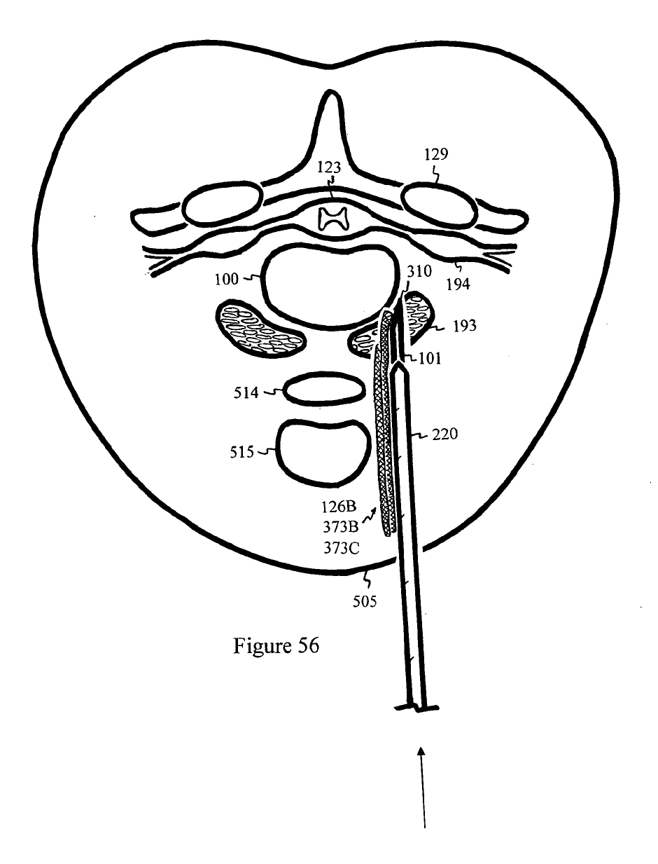

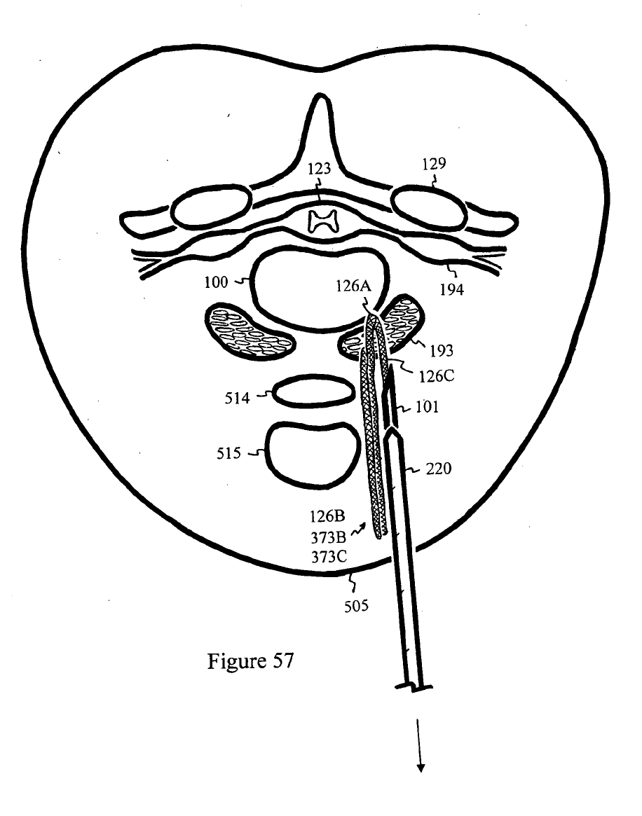

The internal and/or external disc shunt 126, 373 can treat the cervical disc 100 as well. The Quincke tip 310 of the needle 101 is preferred to point away from the esophagus 514 and larynx/trachea 515, as shown in FIG. 56. Cervical discs 100 are thin; the superior 106A and/or inferior 106B diffusion zone can be reached by a single or few spirals of short shunt strands 126B, 126C, 373B, 373C. However, during needle 101 insertion toward the intervertebral disc 100, the proximal ends of the short shunt strands 126B, 126C, 373B, 373C can be under the skin 505 as shown in FIG. 56. If the needle 101 is misguided as shown in FIG. 56, the physician would have to slightly withdraw the needle 101, then bend the proximal portion of the needle 101 above the skin 505 to change penetrating direction of the needle 101 beneath the skin 505. However, the slight withdrawal of the needle 101 would deploy the shunt strands 126B, 126C, 373B, 373C prematurely under the skin 505, as shown in FIG. 57, by pulling or exposing the shunt strand 126C from the lumen 269 of the needle 101.

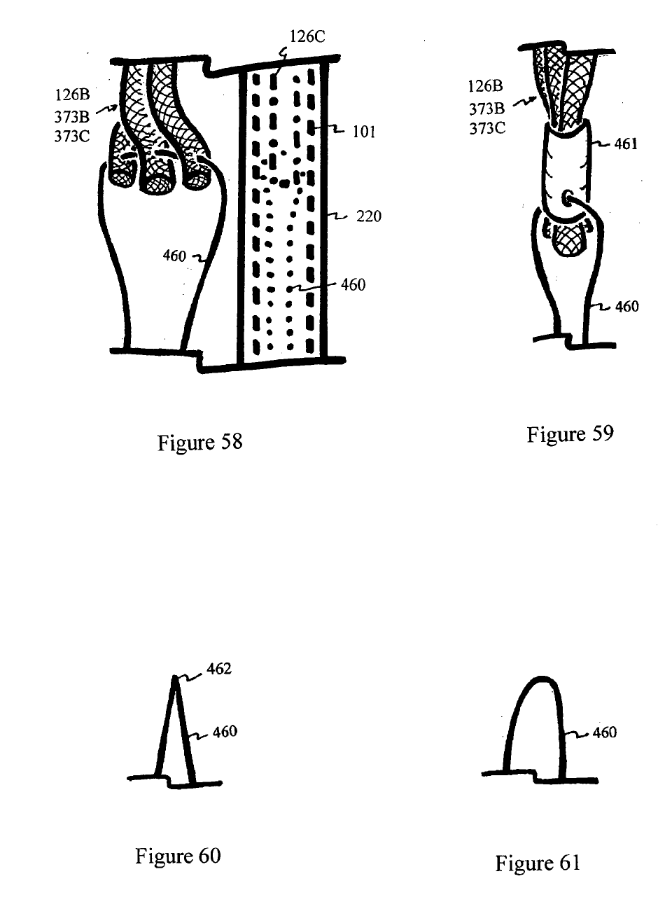

A pull line 460 is threaded through the proximal ends or portions of the shunt strands 126B, 373B, 373C, as shown in FIG. 58. Another pull line 460 can also thread through the proximal portion of the shunt strand 126C within the needle 101. A retainer 461 can be used to hold the shunt strands 126B, 373B, 373C together for attachment to the pull line 460, as shown in FIG. 59. The retainer 461 is made with biocompatible and/or biodegradable material. The pull line 460 is made with a kink-, fold- or crease-resistant material, such as nylon monofilament suture, poly-propylene monofilament suture or other. During tension pulling on the shunt strands 126B, 373B, 373C, a fold or crease 462 would inevitably form on the pull line 460, as depicted in FIG. 60. When tension is released, the fold or crease 462 disappears from the fold-resistant pull line 460, as shown in FIG. 61, to facilitate withdrawal of the pull line 460 from the shunt strands 126B, 373B, 373C under the skin 505.

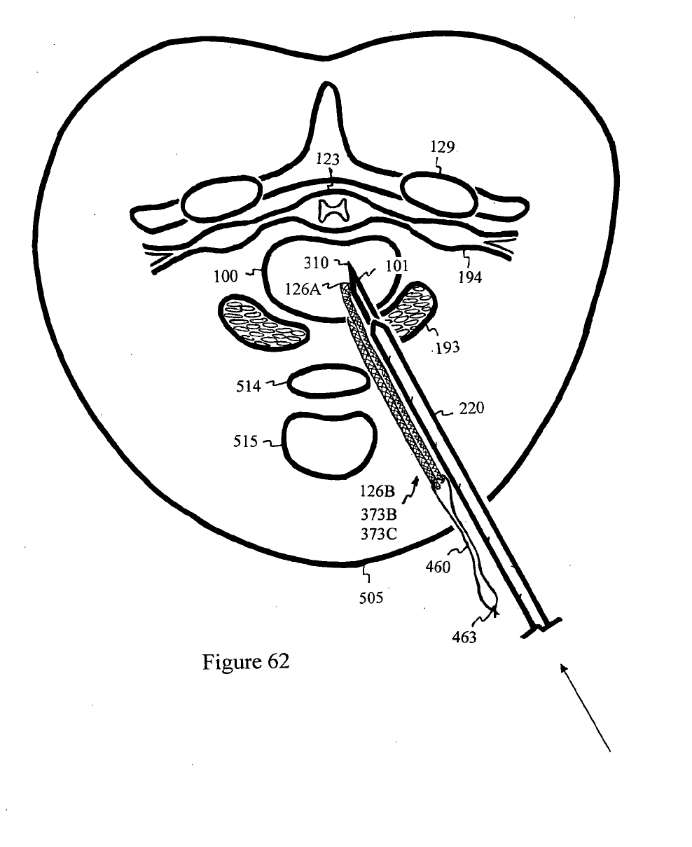

FIG. 62 shows the pull line 460 attached to the shunt strands 126B, 373B, 373C and extending outside the skin 505. The pull line 460 can be a loop, joined by a knot 463 outside the skin 505. If the needle 101 is misguided under fluoroscopic view, as depicted in FIG. 56, tension is applied to the pull line 460 during partial withdrawal of the needle 101. Tension on the pull line 460 keeps the U-section 126A positioned at the distal lumen 269 opening of the withdrawing needle 101. From cadaveric studies and human clinical, the pull line 460 attached to the shunt strands 126B, 373B, 373C is sufficient for partial withdrawal of the needle 101 before re-directing; another pull line 460 attached to the shunt strand 126C within the needle 101 is optional. After sufficient spiraling and delivery of shunt strands 126B, 126C, 373B, 373C within the cervical disc 100 to form internal and/or external disc shunt 126, 373 by the needle 101 and sleeve 220 as shown in FIGS. 15-22, a strand of the fold-resistant pull line 460 is cut next to the knot 463. By holding the knot 463, the pull line 460 is pulled and retrieved from shunt strands 126B, 373B, 373C beneath the skin 505 of the patient. The needle 101 and sleeve 220 are then withdrawn from the patient.

In the United States, average age of patients undergoing back surgery is about 40-45 years old. The internal and/or external disc shunts 126, 373 are preferred to be made with permanent material to provide long-lasting pain relief. A wide range of non-degradable materials can be used to fabricate the shunt strands 126, 373. Polymers, such as Nylon, polytetrafluoroethylene, polypropylene, polyethylene, polyamide, polyester, polyurethane, silicon, poly-ether-ether-ketone, acetal resin, polysulfone, polycarbonate, silk, cotton, or linen are possible candidates. Fiberglass can also be a part of the shunt strands 126, 373 to provide capillarity for transporting nutrients 131 and waste.

Especially for investigative purposes, biodegradable shunts 126, 373 may provide evidence within weeks or months. Since the internal and external disc shunts 126, 373 degrade within months, any unforeseen adverse outcome would be dissipated. If the investigative-degradable disc shunts 126, 373 shows promise, permanent internal and external shunts 126, 373 can then be implanted to provide continuous benefits. The biodegradable shunt strands 126, 373 can be made with polylactate, polyglycolic, poly-lactide-co-glycolide, polycaprolactone, trimethylene carbonate, silk, catgut, collagen, poly-p-dioxanone or combinations of these materials. Other degradable polymers, such as polydioxanone, polyanhydride, trimethylene carbonate, poly-beta-hydroxybutyrate, polyhydroxyvalerate, poly-gama-ethyl-glutamate, poly-DTH-iminocarbonate, poly-bisphenol-A-iminocarbonate, poly-ortho-ester, polycyanoacrylate or polyphosphazene can also be used.

The needle 101, sleeve 220, dip stick 109 and cannula needle 230 can be made with stainless steel, nickel-titanium alloy or other metal or alloy. The needle 101, sleeve 220 and/or cannula needle 230 can be coated with lubricant, tissue sealant, analgesic, antibiotic, radiopaque, magnetic and/or echogenic agents.

The internal and/or external disc shunts 126, 373, can be used as a drug delivery device, delivering oral, intravenous or injectable drugs into the avascular or nearly impenetrable disc 100 to treat infection, inflammation, pain, tumor or other disease. Drugs can be injected into the muscle 193 to be drawn into the external shunted disc 100. Discitis is a painful infection or inflammatory lesion in the intervertebral disc 100 of adults and children (Wenger D R, Bobechko W P, Gilday D L: The spectrum of intervertebral disc-space infection in children, J. Bone Joint Surg. Am., 60:100-108, 1978. Shibayama M, Nagahara M, Kawase G, Fujiwara K, Kawaguchi Y, Mizutani J: New Needle Biopsy Technique for Lumbar Pyogenic Spondylodiscitis, Spine, 1 November, Vol. 35-Issue 23, E1347-E1349, 2010). Due to the avascular nature of the disc 100, oral or intravenous drugs cannot easily reach the bacteria or inflammation within the disc 100. Therefore, discitis is generally difficult to treat. However, the internal and/or external disc shunts 126, 373 can be used as a drug-delivery device. The internal disc shunts 126, 373 draw the systemic drugs through the endplates 105; and the external disc shunts 126, 373 draw the systemic drugs from muscles 193 into the sealed, avascular disc 100. In addition, antibiotics, anti-inflammatory drugs, anesthetics or other drugs can be injected into the muscle 193 near the strands of the external disc shunts 126, 373 to increase drug concentration within the disc 100 to treat discitis or pain. Injection near the external shunt strands 126, 373 is called peri-shunt injection.

Staphylococcus aureus is the most common bacteria found in discitis. The shunt strands 126, 373 can be loaded or coated with an antibiotic, such as nafcillin, cefazolin, dicloxacillin, clindamycin, bactrim, penicillin, mupirocin (bactroban), vancomycin, linezolid, rifampin, sulfamethoxazole-trimethoprim or other, to treat staphylococcus aureus infection. Corynebacterium is also found in discitis. The shunt strands 126, 373 can be loaded or coated with an antibiotic, such as erythromycin, vancomycin, eifampin, penicillin or tetracycline, to treat corynebacterium infection. Other antibiotics, such as cefdinir, metronidazole, tinidazole, cephamandole, latamoxef, cefoperazone, cefmenoxime, furazolidone or other, can also be used to coat the shunt strands 126, 373.

Inflammation in the disc 100 can cause excruciating pain. MRI can show inflammation at the endplates 105, and distinguish inflammatory classification as Modic I, II or III. The disc shunt strands 126, 373 can be coated or loaded with nonsteroidal anti-inflammatory drugs/analgesics. (NSAID), such as aspirin, diflunisal, salsalate, ibuprofen, naproxen, fenoprofen, ketoprofen, flurbiprofen, oxaprozin, indomethacin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, nimesulide, licofelone or other NSAID, to treat inflammation in the disc 100 for pain relief.

The disc shunt strands 126, 373 can also be coated or loaded with steroidal anti-inflammatory drugs/analgesics, such as betamethasone, budesonide, cortisone, dexamethasone, hydrocortisone, methylprednisolone, prednisolone, prednisone, triamcinolone or other steroid, to treat inflammation in the disc 100 for pain relief.

The shunt strands 126, 373 can be loaded or coated with anesthetics, such as procaine, amethocaine, cocaine, lidocaine, prilocaine, bupivacaine, levobupivacaine, ropivacaine, mepivacaine, dibucaine, methohexital, thiopental, diazepam, lorazepam, midazolam, etomidate, ketamine, propofol, alfentanil, fentanyl, remifentanil, sufentanil, buprenorphine, butorphanol, diamorphine, hydromorphone, levophanol, meperidine, methadone, morphine, nalbuphine, oxycodone, oxymorphone, pentazocine or other anesthetic, to provide instant pain relief.

The shunt strands 126, 373 can be loaded or coated with a muscle relaxant, such as succinylcholine, decamethonium, mivacurium, rapacuronium, atracurium, cisatracurium, rocuronium, vecuronium, alcuronium, doxacurium, gallamine, metocurine, pancuronium, pipecuronium, tubocurarine or other relaxant, to relief muscle tension and ache.

The shunt strands 126, 373 can be loaded or coated with buffering agents, such as sodium carbonate, sodium bicarbonate, potassium carbonate, potassium bicarbonate, magnesium carbonate, calcium carbonate, barium carbonate, potassium phosphate, sodium phosphate or other buffering agent, to neutralize lactic acid 162 and spontaneously alleviate pain caused by acid irritation or burn.

The shunt strands 126, 373 can be loaded or coated with alkaline agents, such as magnesium oxide, magnesium hydroxide, sodium hydroxide, potassium hydroxide, barium hydroxide, cesium hydroxide, strontium hydroxide, calcium hydroxide, lithium hydroxide, rubidium hydroxide, neutral amines or other alkaline agent, to neutralize lactic acid 162 and spontaneously alleviate pain caused by acid irritation.

The shunt strands 126, 373 can be loaded or coated with initial supplies of nutrients 131, such as sulfate, glucose, glucuronic acid, galactose, galactosamine, glucosamine, hydroxylysine, hydroxylproline, serine, threonine, chondroitin sulfate, keratan sulfate, hyaluronate, magnesium trisilicate, magnesium mesotrisilicate, magnesium oxide, magnosil, orthosilicic acid, magnesium trisilicate pentahydrate, sodium metasilicate, silanolates, silanol group, sialic acid, silicic acid, boron, boric acid, other mineral, other amino acid or nutrients 131, to enhance or initiate production of sulfated glycosaminoglycans and collagen within the degenerative disc 100.

Oral intake of antidepressants has shown temporary pain reduction or pain tolerance in back pain patients. Anti-depressants can be coated on the shunt strands 126, 373 to treat chronic back pain. The anti-depressant coating may include tricyclic antidepressant, serotonin-reuptake inhibitor, norepinephrine reuptake inhibitor, serotonin-norepinephrine reuptake inhibitor, noradrenergic/serotonergic antidepressants, norepinephrine-dopamine reuptake inhibitor, serotonin reuptake enhancers, norepinephrine-dopamine disinhibitors or monoamine oxidase inhibitor. The antidepressant can be amitriptyline, amitriptylinoxide, butriptyline, clomipramine, demexiptiline, desipramine, dibenzepin, dimethacrine, dosulepin/dothiepin, doxepin, duloxetine, imipramine, imipraminoxide, lofepramine, melitracen, metapramine, nitroxazepine, nortriptyline, noxiptiline, pipofezine, propizepine, protriptyline, quinupramine, amineptine, iprindole, opipramol, tianeptine, trimipramine, or other antidepressant.

Fibrous formation over the internal and/or external shunts 126, 373 may affect the exchange of nutrients 131 and waste between the disc 100 and bodily circulation or muscle 193. Immuno inhibitor can be coated or incorporated into the shunt strands 126, 373 to minimize fibrous formation or tissue response. Examples of immuno inhibitors include but are not limited to: actinomycin-D, aminopterin, azathioprine, chlorambucil, corticosteroids, crosslinked polyethylene glycol, cyclophosphamide, cyclosporin A, 6-mercaptopurine, methylprednisolone, methotrexate, niridazole, oxisuran, paclitaxel, polyethylene glycol, prednisolone, prednisone, procarbazine, prostaglandin, prostaglandin E₁, sirolimus, steroids or other immune suppressant drugs.

The shunt strands 126, 373 can be loaded or coated with a calcium channel blocker for inhibiting activation of neuro-receptor to alleviate pain. The calcium channel blocker can be dihydropyridines, phenylalkylamines, benzothiazepines, magnesium ion, Amlodipine, Felodipine, Isradipine, Lacidipine, Lercanidipine, Nicardipine, Nifedipine, Nimodipine, Nisoldipine, Verapamil, Diltiazem or other calcium channel blocker.

Healthy intervertebral discs 100 are avascular. To ensure avascular conditions, the shunt strands 126, 373 can be incorporated, coated or partially coated with an anti-angiogenic compound. Examples of anti-angiogenic compounds include, but are not limited to, Marimastat from British Biotech [a synthetic inhibitor of matrix metalloproteinases (MMPs)], Bay 12-9566 from Bayer (a synthetic inhibitor of tumor growth), AG3340 from Agouron (a synthetic MMP inhibitor), CGS 27023A from Novartis (a synthetic MMP inhibitor), COL-3 from Collagenex (a synthetic MMP inhibitor, Tetracycline® derivative), Neovastat from Aeterna, Sainte-Foy (a naturally occurring MMP inhibitor), BMS-275291 from Bristol-Myers Squib (a synthetic MMP inhibitor), TNP-470 from TAP Pharmaceuticals, (a synthetic analogue of fumagillin; inhibits endothelial cell growth), Thalidomide from Celgene (targets VEGF, bFGF), Squalamine from Magainin Pharmaceuticals (Extract from dogfish shark liver; inhibits sodium-hydrogen exchanger, NHE3), Combretastatin A-4 (CA4P) from Oxigene, (induction of apoptosis in proliferating endothelial cells), Endostatin collagen XVIII fragment from EntreMed (an inhibition of endothelial cells), Anti-VEGF Antibody from Genentech, [Monoclonal antibody to vascular endothelial growth factor (VEGF)], SU5416 from Sugen (blocks VEGF receptor signaling), SU6668 from Sugen (blocks VEGF, FGF, and EGF receptor signaling), PTK787/ZK 22584 from Novartis (blocks VEGF receptor signaling), Interferon-alpha (inhibition of bFGF and VEGF production), Interferon-alpha (inhibition of bFGF and VEGF production), EMD121974 from Merck, KcgaA (small molecule blocker of integrin present on endothelial cell surface), CAI from NCI (inhibitor of calcium influx), Interleukin-12 from Genetics Institute (Up-regulation of interferon gamma and IP-10), IM862 from Cytran, Avastin, Celebrex, Erbitux, Herceptin, Iressa, Taxol, Velcade, TNP-470, CM101, Carboxyamido-triazole, Anti-neoplastic urinary protein, Isotretionin, Interferon-alpha, Tamoxifen, Tecogalan combrestatin, Squalamine, Cyclophosphamide, Angiostatin, Platelet factor-4, Anginex, Eponemycin, Epoxomicin, Epoxy-β-aminoketone, Antiangiogenic antithrombin III, Canstatin, Cartilage-derived inhibitor, CD59 complement fragment, Fibronectin fragment, Gro-beta, Heparinases, heparin hexasaccharide fragment, Human chorinonic gonadotropin, Interferon (alpha, beta or gamma), Interferon inducible protein (IP-10), Interleukin-12 (IL-12), Kringle 5 (plasminogen fragment), Tissue inhibitors of metalloproteinases, 2-Methoxyestradiol (Panzem), Placental ribonuclease inhibitor, Plasminogen activator inhibitor, Prolactin 16 kD fragment, Retinoids, Tetrahydrocortisol-S, Thrombospondin-1, Transforming growth factor beta, Vasculostatin, and Vasostatin (calreticulin fragment).

In summary, the internal and/or external disc shunt 126, 373 alleviates back pain by (1) drawing nutrients/oxygen/pH buffer 131 into the disc 100, (2) neutralizing lactic acid 162 to alleviate acid burn, (3) converting anaerobic to aerobic conditions to reduce lactic acid 162 production, (4) increasing sulfate incorporation in neutral pH for biosynthesis of glycosaminoglycans. (5) increasing ATP production from aerobic metabolism of sugars to drive biosynthetic reactions in disc 100, (6) bulking up the disc 100 to take load off painful facet joints 129, (7) fortifying the disc 100 to reduce spinal instability and muscle tension, (8) rebuilding disc matrix to increase osmolarity, fluid intake and absorption, (9) re-establishing the swelling pressure to sustain disc 100 compression, (10) regenerating the disc 100 for long term pain relief, and/or (11) delivering systemic drugs in disc 100 to treat discitis.

Unlike many surgical interventions of the spine, benefits of the internal and/or external disc shunts 126, 373 include (1) spinal motion preservation, (2) no tissue removal, (3) reversible by extraction, (4) micro-invasive, (5) out-patient procedure, (6) approved implant material, (7) 15-minutes per disc, (8) long-lasting and no-harm-done, (9) no incision, (10) compatible with drugs, conservative treatment or surgical intervention, if needed, and (11) drug coated shunt if needed to expedite pain relief.

The internal disc shunt device can be used to spiral and pack coiled or spiraled strands 126, 373 into a mucosal wall of a urethra to treat urinary stress incontinence. The strands 126, 373 can be a nylon or polypropylene mono-filament suture, to provide an elastic backboard support within the posterior mucosal wall of the urethra. The coils of spiraled strands 126, 373 in the mucosal wall also serve as a bulking agent, narrowing the urethral lumen opening to enhance or restore sphincteric control of the urethra.

The spiraling device can also be used to spiral and pack strands 126, 373 under skin, especially into an indentation from acne scar or cosmetic defect.

The present invention is broadly claimed that the shunt strands 126, 373 is delivered by a needle and packed into a disc 100, reaching one or both diffusion zones 106A, 106B between 0 and 3 mm from the endplates 105, to draw nutrients/oxygen/pH buffer 131 diffused from capillaries 107 at the endplate 105 into the mid layer of the disc 100. The needle may also contain a sleeve.

Deployment of the spiraled shunt strands 126, 373 from the distal portion of the needle 101 into the disc 100 can be done without the sleeve 220. Annulus 378 of the disc 100 holds or traps the spiraled or knotted shunt strands 126, 373, while the needle 100 is withdrawn to fully deploy the internal and/or external disc shunts 126, 373. Especially for thin cervical discs 100, the spiraled shunt strands 126, 373 from the second position of the needle 101 may be sufficient, reaching one or both diffusion zones 106A, 106B between 0 and 3 mm from the endplates 105, to draw nutrients/oxygen/pH buffer 131 diffused from capillaries 107 at the endplate 105 into the mid layer of the disc 100. This technique for implanting the internal and/or external disc shunts 126, 373 was used in the in-vivo sheep studies, without failed deployment in nearly 100 sheep discs.

It is to be understood that the present invention is by no means limited to the particular constructions disclosed herein and/or shown in the drawings, but also includes any other modification, changes or equivalents within the scope of the claims. Many features have been listed with particular configurations, curvatures, options, and embodiments. Any one or more of the features described may be added to or combined with any of the other embodiments or other standard devices to create alternate combinations and embodiments. The shunt strands 126B, 126C, 373B, 373C can also have a gate to regulate rate and/or direction of flow. It is also possible to connect a pump to the shunt strands 126B, 126C, 373B, 373C to assist the exchange between the disc 100 and the bodily fluid. A pH electrode may be exposed near the tip of the needle 101 to detect the acidity within the disc 100.

It should be clear to one skilled in the art that the current embodiments, materials, constructions, methods, tissues or incision sites are not the only uses for which the invention may be used. Different materials, constructions, methods or designs for various sections 126A, 373A and end strands 126B, 126C, 373B, 373C can be substituted and used. The internal and/or external disc shunt 126, 373 can be called a conduit, wick, sponge or absorbent. Nothing in the preceding description should be taken to limit the scope of the present invention. The full scope of the invention is to be determined by the appended claims. For clarification in claims, sheath is a tubular member. Spiraled shunt strand can be called a spool of strand or spool shunt.

What is claimed is:

1. A device for treatment of an intervertebral disc, said device comprising:
a needle comprising an outer wall, a distal portion and a proximal portion, a longitudinal lumen, wherein said distal portion comprises a beveled tip,
a sleeve sized and configured to retain said needle, wherein said sleeve comprises a distal end, and wherein said distal end further comprises at least one snagging point,
wherein said sleeve is movable longitudinally along said needle, wherein said at least one snagging point maintains a substantially fixed distance from said outer wall during longitudinal movement of said sleeve,
a first shunt having a first end strand and a second end strand, wherein at least a portion of said first end strand is located in said longitudinal lumen, and at least a portion of said second end strand is outside said needle and sleeve,
wherein said first shunt comprises an outer diameter or thickness, wherein said outer diameter is larger than said substantially fixed distance from said outer wall,
and wherein said distal portion, said at least one snagging point and said second end are sized and configured to enter the intervertebral disc.

2. The device of claim 1, wherein said needle is movable between a first position and a second position,
wherein in said first position, said second end strand is generally linear outside said distal portion of said needle,
and wherein in said second position, said second end strand is formed into a spiraled shunt strand around said distal portion.

3. The device of claim 2, wherein said first position is capable of being converted to said second position by rotating or twisting said needle.

4. The device of claim 2, wherein said at least one snagging point is movable between a position one and a position two,
wherein in said position one, said at least one snagging point is located proximally to said beveled tip,
and wherein in said position two, said at least one snagging point is substantially level or even with said beveled tip, thereby dislodging said spiraled shunt strand from said distal portion of said needle into the intervertebral disc.

5. The device of claim 4, wherein the intervertebral disc comprises a superior and an inferior endplate diffusing nutrients, oxygen and pH buffer from capillaries in adjacent vertebral bodies,
wherein said spiraled shunt strand is located between 0 and 3 mm from at least one of said superior and inferior endplates, thereby reaching and drawing said nutrients, oxygen and pH buffer into a mid-layer of the intervertebral disc.

6. A device for treatment of an intervertebral disc, said device comprising:
a needle comprising an outer wall, a distal portion and a proximal portion, wherein said distal portion further comprises a beveled tip,
a first shunt comprising a first end strand and a second end strand,
wherein said needle is movable between a first position and a second position,
wherein in said first position, said second end strand is generally linear outside said distal portion of said needle,
wherein in said second position, said second end strand is formed into a spiraled shunt strand around said distal portion,
wherein the intervertebral disc comprises a superior and an inferior endplate diffusing nutrients, oxygen and pH buffer from capillaries in adjacent vertebral bodies,
and wherein said spiraled shunt strand is located between 0 and 3 mm from at least one of said superior and inferior endplates, thereby reaching and drawing said nutrients, oxygen and pH buffer into a mid-layer of the intervertebral disc.

7. The device of claim 5 or 6 further comprises a second shunt attaching to said first shunt.

8. The device of claim 5, 6 or 7 wherein said pH buffer neutralizes lactic acid, thereby reducing acid burn and pain.

9. The device of claim 5, 6 or 7, wherein said spiraled shunt strand forms a bulking agent within the intervertebral disc, thereby elevating height of the intervertebral disc and shifting compressive load from facet joints to the intervertebral disc for reducing strain and pain in said facet joints.

10. The device of claim 5, 6 or 7, wherein said spiraled shunt strand forms a filling in the intervertebral disc, thereby stabilizing the intervertebral disc to reduce spinal instability.

11. The device of claim 5, 6 or 7, wherein at least one of said first end strand, second end strand and second shunt extends from said spiraled shunt strand into a muscle or bodily circulation outside the intervertebral disc, thereby drawing nutrients, oxygen and pH buffer from said muscle or bodily circulation into the intervertebral disc.

12. The device of claim 5 or 6, wherein said first shunt further comprises a U-section locatable between said first end strand and said second end strand.

13. The device of claim 7 or 12 further comprising a rigid cannula needle,
wherein said rigid cannula needle comprises a main lumen,
wherein said needle is elastically curved,
wherein said elastically curved needle is resiliently straightened within said main lumen,
wherein said second end strand is outside said rigid cannula needle,
and wherein said U-section is located at a distal opening of said main lumen.

14. The device of claim 13, wherein said rigid cannula needle further comprises a guide wire lumen.

15. The device of claim 7 or 12, wherein said needle further comprises a guide wire lumen.

16. The device of claim 7 or 12, wherein said needle further comprises an inner wall in said distal portion,
and wherein at least a portion of said inner wall is dull thereby minimizing damage to said U-section.

17. The device of claim 13, wherein said distal opening of said main lumen further comprises an inner wall,
and wherein at least a portion of said inner wall is dull thereby minimizing damage to said U-section.

18. The device of claim 7 or 12 further comprises a dip stick insertable into said needle for detecting depth of said first end strand.

19. The device of claim 7 or 12 further comprising a pull line attaching said first shunt.

20. The device of claim 12, wherein said first shunt has water absorbing capability by gaining weight between about 10% and about 500% at ambient temperature and pressure.

21. The device of claim 7 or 12, wherein said first shunt has pore sizes between about 1 nano-meter and about 200 micro-meter.

22. The device of claim 7 or 12, wherein said first shunt is characterized by having water contact angle between about 0 and about 60 degree at in ambient temperature and pressure.

23. The device of claim 7 or 12, wherein said first shunt is characterized by having capillary action for drawing saline between about 0.5 and about 120 cm in ambient temperature and pressure.

24. The device of claim 7 or 12, wherein said first shunt comprises cells.

25. The device of claim 7 or 12, wherein said first shunt comprises a coating comprising an antibiotic.

26. The device of claim 7 or 12, wherein said first shunt comprises a coating comprising an anti-inflammatory drug.

27. The device of claim 7 or 12, wherein said first shunt comprises a coating comprising an anesthetic.

28. The device of claim 7 or 12, wherein said first shunt comprises a coating comprising a muscle relaxant.

29. The device of claim 7 or 12, wherein said first shunt comprises a coating comprising a buffering agent.

30. The device of claim 7 or 12, wherein said first shunt comprises a coating comprising an alkaline agent.

31. The device of claim 7 or 12, wherein said first shunt comprises a coating comprising a nutrient.

32. The device of claim 7 or 12, wherein said first shunt comprises a coating comprising an anti-depressant.

33. The device of claim 7 or 12, wherein said first shunt comprises a coating comprising an immuno inhibitor.

34. The device of claim 7 or 12, wherein said first shunt comprises a coating comprising a calcium channel blocker.

35. The device of claim 7 or 12, wherein said first shunt comprises a coating comprising an anti-angiogenic compound.

36. The device of claim 7 or 12, wherein said first shunt comprises fabric filaments.

37. (canceled)

38. (canceled)
